(19) United States
(12) Patent Application Publication
(10) Pub. No.: US 2005/0089853 A1
(43) Pub. Date: Apr. 28, 2005

(54) Method

(76) Inventor: Anne Spurkland, Oslo (NO)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/474,444

(22) PCT Filed: Apr. 3, 2002
(86) PCT No.: PCT/GB02/01526

(30) Foreign Application Priority Data
Apr. 3, 2001 (GB) 010349.2

(51) Int. Cl. C12Q 1/68; C12N 15/00

(52) U.S. Cl. 435/6; 435/320.1

(57) ABSTRACT

The present invention provides an improved method of separation of alleles in a sample (sample alleles), involving the use of a reference allele and further comprising the use of double stranded sample alleles and/or the use of said reference allele in a double stranded form wherein one of the strands of the double stranded alleles present has been labelled so as to allow specific digestion of one of the strands. In preferred embodiments the label is a 5′-phosphorylated group and the digestion is carried out using λ-exonuclease. Use of this method in genotyping and/or diagnosis and kits for use in such methods of allele separation and genotyping are also included.

The present invention relates to an improved method of allele separation comprising the use of a modified reference allele. The present invention further relates to the use of this method in the genomic typing of gene loci containing one or more polymorphic positions. The invention further relates to modified reference alleles for use in such methods and kits comprising such modified reference alleles.

Determination of the alleles present at a given locus in heterozygous individuals (i.e. genomic or allele typing) represents an important challenge to geneticists. The possibility to perform in vitro amplification of the alleles before subsequent determination of the polymorphic positions in the amplicon has allowed development of rapid genomic typing methods of a number of polymorphic loci. However; most methods are based on simultaneous sequence determination of the two alleles present in any given sample. For highly polymorphic loci, such as the HLA class I loci, this yields a high frequency (10-20%) of samples with ambiguous typing results.

To resolve such ambiguities, the alleles present in the sample can be amplified separately in vitro, using primers specific for given polymorphic positions observed in the sample, and thereafter reanalysed using the chosen technique. Alternatively, specific amplification procedures are applied at the start, without any prior knowledge of the alleles present, and the resulting amplicons are analysed thereafter.

The possibility of ambiguous typing results and the cumbersome methods necessary to resolve the ambiguities, make typing of highly polymorphic alleles costly both in terms of workforce, time and reagents. Also, as automat sequencing has become more widely used for PCR based typing of DNA and RNA species, a particular problem has been to ensure reliable identification of heterozygous positions in the sequence. This problem has very much retarded development of robust and simple commercially available systems for genomic typing based on automatic sequencing.

To overcome the problems associated with direct sequencing of the alleles present in a sample, techniques have been developed which allow the physical separation of alleles before the determination of the polymorphic positions present in each of the two alleles. Such techniques are based on the melting properties of the alleles (e.g. denaturating gradient gel electrophoresis (DGGE)), or based on the conformation of alleles (e.g. by single strand conformation polymorphism (SSCP), or by the formulation of heteroduplexes via the use of a reference strand (e.g. reference strand mediated conformation analysis (RSCA) or complementary strand analysis (CSA)). These methods have several limitations.

The DGGE method is dependent on the melting properties of the particular sequence. There is also a limitation to the length of sequence that may be separated in one experiment. Thus, typically 500-600 bases may be separated using the DGGE method depending on the sequence. In addition, single nucleotide polymorphisms may not be detected by this technique.

The SSCP method is highly dependent on the composition of the separating medium, i.e. the acrylamide or ion concentration in the resolving PAGE gel used. For specific banding to occur, all the molecules of each of the single stranded alleles must attain the same single stranded conformation in the electrophoresis, which is not always the case. Thus, often several more bands than expected may be observed at a given condition for a given set of two alleles thereby causing difficulty in interpretation and reproducibility of results. In addition, SSCP resolution is limited to 300 bp single stranded DNA.

The RSCA method is dependent on the use of a reference DNA (typically one rare allele of the locus of interest) to form heteroduplexes with the alleles to be separated. It has been shown that this method allows the separation of gene fragments of up to 1 kb which only differ by one nucleotide. However, for downstream analysis of the allele sequences, the separated alleles are still “heterozygous” as the reference DNA is also present in the heteroduplex. Moreover, heterozygous samples carrying one allele corresponding to the reference DNA will appear as false homozygotes in the RSCA assay. In order to avoid this possibility, two different reference DNA molecules must be used. The use of two different reference molecules per sample however results in interpretation becoming complicated. Thus, in general, two different reference DNA molecules are used in two different experiments.

Relatively recently a modification of the RSCA method of physical separation of alleles was developed which overcomes these problems (Arguello et al., 1997, Nucleic Acids Research, 25(11), 2236-2238). This technique involved the introduction of a mutation in the primer binding regions of the reference DNA fragments which was designed to prevent the sample allele specific primers being able to hybridise with the reference fragments, thereby allowing the sample alleles and not the reference alleles of heteroduplexes to be specifically amplified by PCR and then analysed further. In addition, the presence of this modification meant that the reference DNA no longer corresponded to a rare sample allele at the locus in question and thus, the problem with false homozygote results was reduced.

In both the original and modified RSCA methods, when reference DNA is mixed with heterogenous sample DNA a number of different completes may be formed, i.e. heteroduplexes between the reference DNA and the sample DNA (up to 4 molecules) as well as heteroduplexes between the sample alleles (up to 2 molecules). In addition three different homoduplexes may be formed (2 of sample alleles and one of reference alleles). As the genotyping relies on the manipulation and analysis of the heteroduplexes formed between the reference DNA and the sample DNA, the presence of homoduplexes and also the heteroduplexes between the sample alleles is undesirable and can result in confused results, especially when the undesired homoduplexes and heteroduplexes do not physically separate from the desired heteroduplexes to a sufficient extent to enable the desired heteroduplexes to be easily separated and subjected to further analysis.

One way to reduce such problems is to remove from the mixture to be analysed one of the strands of the sample alleles (i.e. remove either the coding or the complementary strands of the sample alleles) and to remove the opposite strand from the reference DNA. This can be done for example by labelling the strands it is desired to remove with a label which allows them to bind some kind of solid support, e.g. by labelling the strands with biotin and then effecting the separation using a streptavidin coated solid support, e.g. streptavidin coated magnetic beads. Such methods of labelling and separating particular strands of DNA molecules and variations thereof are well known and described in the art and any of these methods may be used. The removal of one of the strands from each of the sample and reference alleles means that when the sample and reference DNA are remixed only heteroduplexes should be formed. The disadvantage of this solution is that the additional procedure of labelling and separating the strands of both the sample DNA and the reference DNA in this manner is laborious and costly and, in addition, is unlikely to be completely effective. Thus, it would be an advantage to eliminate or simplify this additional step.

The present invention overcomes the problem of the undesired homoduplexes and heteroduplexes interfering with the physical separation of the desired heteroduplexes formed between the sample and the reference DNA and, furthermore, overcomes this problem without the need for the extra steps of labelling and separating one of the strands of both the sample and the reference DNA before heteroduplexes are formed. However, a new improved method of generating single stranded DNA has also been developed to aid desired heteroduplex formation. Thus, the present invention provides improved methods of allele separation.

One of the ways the present invention achieves this is by using a modified reference DNA which has been altered or mutated so that heteroduplexes formed between sample and reference alleles are protected from digestion by one or more endonucleases, e.g. restriction enzymes, whereas any undesired homoduplexes of sample or reference alleles or heteroduplexes formed between sample alleles will be digested by the same endonucleases, e.g. restriction enzyme(s). In this way the undesired duplexes will be cut into two or more fragments which are significantly smaller in size than the full length desired heteroduplexes and thus migrate significantly differently on whatever separating medium is used for allele separation, thereby enabling the desired heteroduplexes to be easily identified and, if required, manipulated and analysed further.

The invention is especially well suited for the typing of loci containing several possible polymorphic positions within the same amplicon. As will be described more fully below, the invention allows for reliable, fully automated genomic typing of heterozygous samples, using equipment that is already developed.

Thus in one aspect the present invention provides an improved method of separation of alleles in a sample, comprising the use of a modified reference allele, wherein said modified reference allele comprises one or more modifications (e.g. mutations) such that heteroduplexes formed between said reference allele and an allele present in the sample (sample allele) will be resistant to digestion with one or more endonuclease enzymes, preferably restriction enzymes, which will digest homoduplexes or heteroduplexes of said sample alleles or homoduplexes of said reference alleles. The various homoduplexes or heteroduplexes which are susceptible to digestion will of course only be digested if they are present in the sample. For example, as will become clear below, homoduplexes of reference alleles will not be present if the modified reference allele is added in a single stranded form.

Viewed alternatively the present invention provides an improved method of separation of alleles in a sample, comprising the use of a modified reference allele and one or more endonuclease enzymes, wherein said modified reference allele comprises one or more modifications (e.g. mutations) such that the said endonuclease enzymes will cut at least once all duplexed molecules in the sample except heteroduplexes formed between the reference allele and an allele from the sample (sample allele).

In a further aspect the invention provides a method of separation of alleles in a sample, comprising the use of a modified reference allele, wherein said modified reference allele comprises one or more modifications (e.g. mutations) such that heteroduplexes formed between said reference allele and an allele present in the sample (sample allele) will be resistant to digestion with one or more endonuclease enzymes which will digest homoduplexes or heteroduplexes of said sample alleles. In such embodiments homoduplexes of the reference allele are either not present (e.g. if the modified reference allele is added in a single stranded form) or are of a structure or conformation such that they separate sufficiently differently to the heteroduplexes formed between the sample and reference alleles so as not to be problematic to or interfere with the overall allele separation method.

Any endonuclease enzyme which does not digest, disrupt, cleave or cut heteroduplexes formed between reference alleles and sample alleles may be used in these methods. Preferably an endonuclease enzyme which can selectively digest, disrupt, cleave or cut any homoduplexes or heteroduplexes of said sample alleles and optionally also homoduplexes of said reference alleles present in the sample, but not digest, disrupt, cleave or cut heteroduplexes between reference alleles and sample alleles present is used in these methods. Preferably the endonuclease enzymes used for example induce cleavage at a particular site in a nucleic acid molecule, which site is present in the unwanted duplexes but not present in the heteroduplexes formed between reference and sample alleles. Alternatively, the endonuclease enzymes may induce cleavage at one or more particular modified bases e.g. a synthetically modified nucleotide or methylated nucleotide, e.g. methylated C residues which are susceptible to cleavage by methyl specific endonucleases. Preferably the endonuclease enzymes used are restriction enzymes. Thus in all the embodiments of the invention where the use of restriction enzymes is described, other types of endonuclease enzymes with appropriate activity could be used.

By using this method, heteroduplexes formed between sample and reference alleles will be protected from endonuclease, e.g. restriction enzyme digestion and occur as one or more discrete bands or peaks (depending on the method of separation used) well separated from the remaining nucleic acids in the sample.

In a preferred embodiment of the invention only one of the strands of the modified reference allele is added to the sample as opposed to the addition of the modified reference allele in a double stranded form. This results in a further simplification of the band or peak pattern and hence further improves the separation of the alleles. In such embodiments after endonuclease, e.g. restriction enzyme digestion the heteroduplexes appear as one or two bands well separated from the remaining nucleic acid molecules in the sample. In this embodiment the presence of one band indicates a homozygous sample and the presence of two bands a heterozygous sample. In this embodiment digestion of homoduplexes of the reference alleles will not take place as they will not be present in the mixture due to the fact that the reference allele is added in a single stranded form. Thus, only digestion of homoduplexes or heteroduplexes of sample alleles will take place.

In a further preferred embodiment of the invention either or both of the sample alleles and reference alleles are added or contained in the sample in a double stranded form and rendered single stranded by the addition of an appropriate enzyme or agent which is specific for one of the strands of the double stranded sample alleles and reference alleles and digests said strand. Such specificity for one of the two strands can be obtained by the appropriate labelling of said strand. For example, a preferred enzyme for use in this regard is λ exonuclease which causes the specific digestion of strands of nucleic acid which are phosphorylated at the 5′ end. Such phosphorylated labels at the 5′ ends of one of the strands of double stranded nucleic acid can easily be introduced by methods known in the art, such as for example using a PCR primer with a phosphorylated 5′ end for the amplification of the sample alleles and/or the reference alleles.

This method whereby one of the strands or double stranded alleles are digested can be used alone to aid heteroduplex formation or it can be used in conjunction with the use of a restriction enzyme and a modified reference allele as described herein.

Thus, in a yet further embodiment of the invention the methods of allele separation as described herein using a modified reference allele and one or more endonuclease enzymes further comprise the use of a second enzyme specific for appropriately labelled strands of double stranded nucleic acid. In a preferred embodiment this second enzyme is λ exonuclease which is specific for nucleic acid strands containing a phosphorylated 5′ end. Said second enzyme (e.g. λ exonuclease) may be added before, at or around the same time as the appropriate restriction enzymes described herein. Preferably however said second enzyme is added before the induction of heteroduplex formation, and the appropriate restriction enzymes described herein are added after the induction of heteroduplexes. In other words an enzyme such as λ exonuclease is used in combination with restriction digestion to aid the formation of heteroduplexes. In such methods the formation of heteroduplexes is enriched by the action of the restriction enzyme as described above to digest undesired homoduplexes or heteroduplexes of the sample alleles and homoduplexes of the modified reference alleles and further enriched by the action of λ exonuclease which digests undesired nucleic acid strands which have been labelled with a 5′ phosphorylated group.

A yet further aspect of the invention provides an improved method of separation of alleles in a sample (sample alleles), involving the use of a reference allele and further comprising the use of double stranded sample alleles and/or the use of said reference allele in a double stranded form wherein one of the strands of the double stranded alleles present has been labelled so as to allow specific digestion of one of the strands. A preferred label is a 5′ phosphorylated group which leads to the labelled strand being digested by the enzyme λ exonuclease as described above. “Modified reference alleles” as defined herein can also be used in such methods.

In such methods, and indeed in the methods where specific strand digestion, e.g. λ exonuclease digestion is used in combination with endonuclease, e.g. restriction enzyme digestion, in the cases where both the sample and reference alleles are provided in a double stranded form, generally either the sense or the antisense strand of the sample alleles are labelled with a 5′ phosphorylated group and the other/opposing strand of the double stranded reference alleles are labelled with a 5′ phosphorylated group. The λ exonuclease when added then digests the phosphorylated strands leaving the opposing strands of the reference and sample alleles available to form heteroduplexes (under appropriate conditions) which can then be separated by the methods described herein. Alternatively only one of the sample and reference alleles are supplied in a double stranded form with one of the strands phosphorylated at the 5′ end. λ exonuclease action then leads to the formation of single stranded nucleic acid via digestion of the phosphorylated strand. Also part of this mixture in a single stranded form (or added to this mixture in a single stranded form) is the appropriate opposing strand of sample or reference alleles to the non digested strand of the relevant allele, which thereby results in the formation of heteroduplexes (under appropriate conditions) which can be separated by the methods described herein. For example, if the sample allele is provided in a double stranded form and the sense strand is phosphorylated and thereby digested by the λ exonuclease, single stranded sense reference allele also forms part of the mixture, etc.

Methods of heteroduplex formation comprising the use of λ exonuclease and/or restriction enzymes as described herein form yet further aspects of the present invention.

“Sample allele” as used herein refers to a nucleic acid molecule and generally a DNA molecule which corresponds to one of the alternative forms of a specified gene at a particular locus. The DNA molecule thus comprises or includes a region (i.e. a nucleotide sequence) in which one or more allelic variations or polymorphisms may exist. Such nucleic acid molecules may thus include larger stretches or parts or pieces (portions) of nucleic acid which comprise the whole of a particular gene, or more than one gene, or may include molecules comprising a part (or portion or fragment) of a gene containing or corresponding to a region of allelic variation (i.e. a region of said gene where allelic variation or polymorphism may occur). Alternatively, the allelic variation (or “allele” as such) may occur at any locus within a nucleic acid (e.g. genomic DNA) and the sample allele may thus be any nucleic acid molecule which contains or comprises a region containing or comprising such a locus. A single gene may have alternative forms (polymorphisms) with one or more (e.g. two, three or more) alleles. Different genes may exist in nature in many allelic forms and any one of these alleles is included within the term “sample allele” for a particular gene. This term includes alternative forms of a gene in genes where there is no “wild-type” form of the gene, as for example in the HLA alleles, and also (as well as the “wild type” sequence itself) includes the alternative forms of a gene at loci where there is a “wild-type” genetic sequence and this wild type sequence is sometimes altered, for example in certain disease states. Alleles for a particular gene may differ from each other by only one or more nucleotides (by e.g. substitution, addition or deletion of nucleotides) or may have more substantial nucleotide substitutions, additions or deletions. This will depend on the particular gene concerned. Also, as mentioned above, alleles may differ from each other at a number of different sites in the gene, i.e. there may be multiple (i.e. two or more) polymorphic sites. The term “sample allele” may refer to the particular nucleic acid sequence of the whole gene (and any of its alleles), or may refer to a fragment of the gene in which the allelic variant residues are found.

Although a particular gene or part thereof may have a large number of allelic forms throughout a population as a whole, it will be appreciated that in a particular sample derived from one genomic source (e.g. a particular patient) a maximum of two different forms of the allele will be present at the DNA level. If two different forms of allele are present in a sample the sample is termed heterozygous and if only one form of allele is present the sample is termed homozygous.

“reference allele” as used herein refers to a nucleic acid molecule and preferably a DNA molecule which is capable of forming a heteroduplex with one or more of the sample alleles for a particular gene and which heteroduplex has a physical conformation different from a homoduplex of the sample alleles or a heteroduplex of the sample alleles or a homoduplex of the reference allele such that the heteroduplexes formed between the sample and reference alleles can be physically separated from any homoduplexes formed and also any sample allele heteroduplexes in an appropriate separation medium. Preferably the reference alleles are capable of forming heteroduplexes with a plurality of sample alleles and most preferably with all the possible sample alleles for a particular gene. It will be appreciated however that in some cases it will not be possible for a single reference allele to be designed which will form heteroduplexes with all the possible sample alleles. Moreover, it will often be difficult to design a single reference allele which will form heteroduplexes with sample alleles which all have sufficiently different migration patterns to enable separation to take place. This is especially the case for a gene which can exist in many different allelic forms. In some situations therefore, more than one reference allele may be used in one or several different reactions to analyse a particular sample.

In order to be able to form such heteroduplexes with the sample alleles the sequences of reference alleles are generally very similar, i.e. share a high degree of sequence identity, to the sample alleles which they are desired to detect, and generally correspond to all or part of a rare allele of the sample allele they are designed to detect. If appropriate, the reference alleles may correspond to all or part of a wild type allele of the gene they are designed to detect. Preferably the reference alleles do not correspond to a rare sample allele or a wild type allele which is found in the genomic DNA in vivo but are recombinant, synthetic or partially synthetic molecules which contain some other modification/mutation which is not found in vivo (such molecules are referred to herein as “modified reference alleles”). These “modified reference alleles” (also referred to herein as “modified reference probes” or “reference probes”) essentially have all the properties of the reference alleles as defined above, except for the fact that they do not correspond to alleles found in vivo, by virtue of the additional modification(s) (i.e. mutation or mutations) which are present.

Such modifications/mutations found in the modified reference alleles can be of any type which can induce mismatch between a sample and a reference allele and include the substitution, addition, and/or deletion of one or more nucleotides at one or more positions in the nucleic acid molecule. Other examples of modifications include the introduction of synthetically modified nucleotides or methylated nucleotides. Such modifications/mutations can be introduced by methods well known and described in the art.

Any number of mutations/modifications can be included in the reference allele compared to the sample alleles providing that the similarities between the two are such that hybridisation or annealing of the sample and reference alleles can occur, thereby leading to the formation of a heteroduplex between a sample and a reference allele. For example, one mutation/change in nucleotide sequence may be sufficient for reference alleles to function in this way. Alternatively more modifications/mutations, e.g. up to 10 or 20 or 30 can be used. In preferred embodiments of the invention at least 1%, for example between 1% and 10% and even up to 20% or up to 30% of the total number of bases making up the reference allele are modified/mutated to form the modified reference alleles.

Viewed another way, preferred modified reference alleles are designed so that at least 1%, for example between 1% and 10% or even up to 20% or up to 30% of the total bases making up the modified reference allele form mismatches with the various sample alleles when heteroduplexes are formed. These mismatches may be generated by the introduction of mutated or otherwise modified bases into the reference allele and/or may be generated automatically due to inherent sequence differences between the particular sample alleles in the sample and the mutated reference allele selected. Thus, the number of additional modifications/mutations that need to be introduced into the reference alleles for use in the invention may vary depending on the number of inherent mismatches between the reference allele and the sample alleles. Generally up to 10, 20 or 30 additional modifications/mutations may be introduced or at least 1%, e.g. between 1% and 10% and even up to 20% or up to 30% of the bases of the reference allele may be modified/mutated as described above.

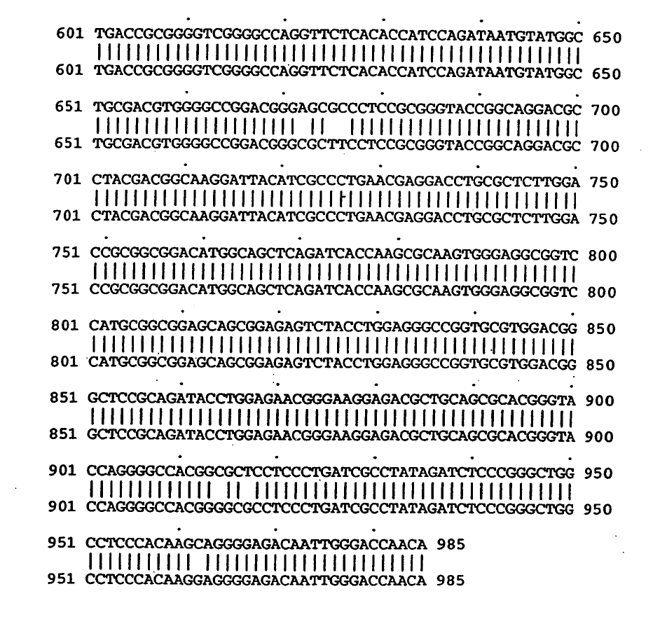

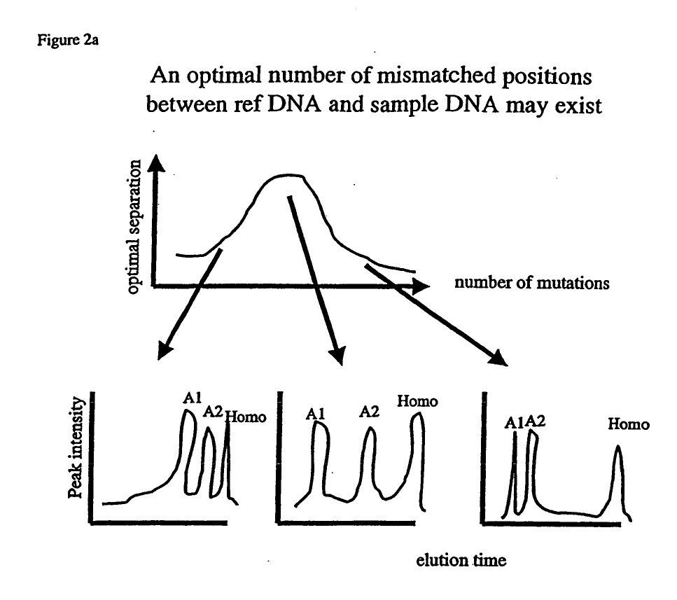

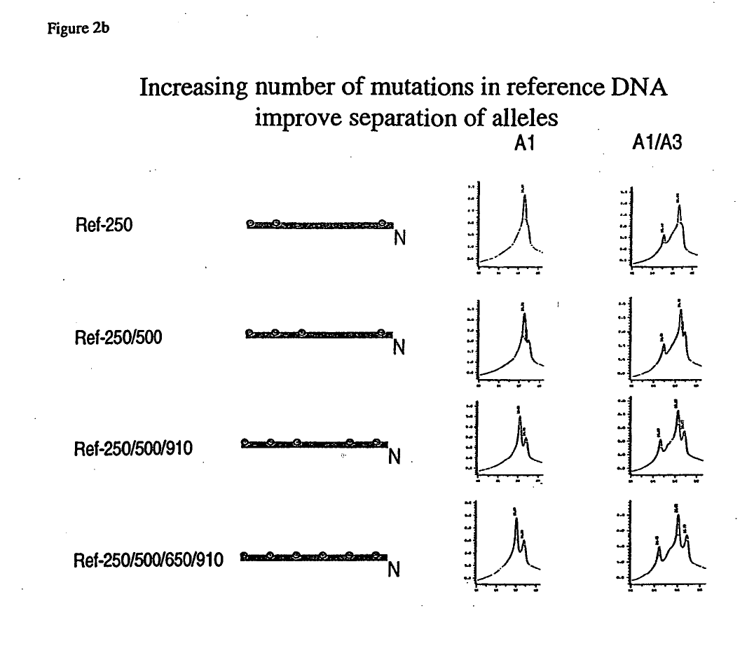

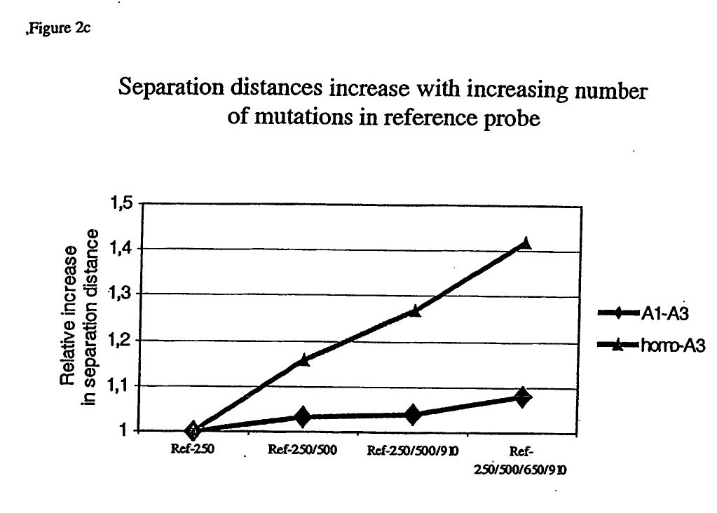

It is expected that there is an optimum percentage of mutations/mismatches which gives the best separation for a particular allele and that a number of mutations/mismatches above or below this value will give less efficient separation (see FIG. 2a). Thus, the above percentage values are only approximations and it would be well within the bounds of a skilled man to elucidate the number of mutations/mismatches which can result in heteroduplex formation.

In the methods of allele separation of the present invention involving the use of endonucleases, e.g. restriction enzymes the “modified reference alleles” comprise mutations/base alterations or modifications such that heteroduplexes formed between said reference alleles and an allele from the sample (sample allele) will be resistant to digestion with one or more endonucleases, e.g. restriction enzymes which can digest homoduplexes or heteroduplexes of said sample alleles or homoduplexes of said reference alleles (if present). These modified reference alleles are generally formed by the removal of restriction sites for one or more restriction enzymes which cut at least once in the particular wild type gene allele/sample allele and the addition or introduction at different locations of at least one site for the same restriction enzyme or enzymes. Preferably said restriction sites are deleted and introduced at sites in the conserved regions of the genes in question, i.e. sites which are not subjected to allelic variation. Designing the reference alleles in this way has the effect that the addition of the selected restriction enzyme(s) to a sample containing any undesired sample allele homoduplexes or heteroduplexes, or reference allele homoduplexes, together with the desired sample/reference allele heteroduplexes, will result in the digestion of sample allele heteroduplexes and homoduplexes (due to the original restriction enzyme sites being present in an unaltered form), the digestion of reference allele homoduplexes (due to the newly introduced restriction enzyme sites for the enzyme(s) in question), but the protection from digestion of any sample/reference allele heteroduplexes as the pairing of the unaltered sample allele with the modified reference allele will not allow any complete restriction sites to form.

The above discussed mutations or alterations or modifications introduced into the modified reference allele can be of any type which are capable of inhibiting endonuclease, e.g. restriction enzyme digestion of desired reference allele/sample allele heteroduplexes but preferably still allow digestion of reference allele homoduplexes (if present) or sample allele homoduplexes or heteroduplexes. Examples include the substitution, addition, and/or deletion of one or more nucleotides at one or more positions in the nucleic acid molecule. Other examples of modifications include the introduction of synthetically modified nucleotides or methylated nucleotides, in particular methylated C residues.

It is important to note that the above described modified reference alleles which can be used in embodiments of the invention involving the use of endonucleases, e.g. restriction enzymes, may also be used as reference alleles in the methods of allele separation of the invention which do not involve the use of restriction enzymes, e.g. in the methods wherein one of the strands of the reference alleles has been labelled so as to allow specific digestion of one of the strands, e.g. by λ exonuclease.

“Homoduplex” as used herein refers to a duplex structure (i.e. a structure made up of two strands of nucleic acid) in which the strands are directly complementary to each other with no mismatches. Examples of homoduplexes are duplexes formed between one of the strands (e.g. the coding strand) of a sample allele and the other/opposite strand (e.g. non-coding strand) of the same sample allele or duplexes formed between one of the strands (e.g. the coding strand) of the reference allele and the other/opposite strand (e.g. non-coding strand) of the reference allele.

“Heteroduplex” as used herein refers to a duplex structure (i.e. a structure made up of two nucleic acid strands) in which the strands are sufficiently similar to anneal/hybridise together to form a duplex molecule, but contain some mismatched oligonucleotide pairs. Examples of heteroduplexes are duplexes formed between one of the strands of the sample allele and the opposite strand of the reference allele or duplexes formed between one of the strands (e.g. the coding strand) of one sample allele and the other/opposite strand (e.g. the non-coding strand) of the other sample allele present in the sample. The sample allele heteroduplexes will of course only form if there are two different alleles present, i.e. the sample is heterozygous. If only one form of the allele is present then homoduplexes (i.e. duplexes with no mismatches) will be formed.

It is the mismatches formed between the sample and reference alleles (or indeed between the two different sample alleles) which produce the difference in migration in a separation medium in comparison to the migration seen with the homoduplexes. The conformation of nucleic acid duplexes are very sensitive to such mismatches and thus the migration in a separating medium will vary according to the nature of the mismatch. In this way alleles may be separated from each other (as they form different heteroduplexes) and from the homoduplexes of reference alleles or sample alleles and from heteroduplexes of sample alleles.

Clearly in samples where many types of homo and heteroduplexes are present separation of alleles can become quite complex and difficult, especially where one or more species of duplex shows relatively similar migration patterns. It is for this reason that the prior art methods are generally based on the preparation and use of sample and reference alleles in a single stranded form, or the use of a reference allele where one strand is detectably labelled (Arguello et al., supra). As discussed above, the preparation of both sample and references alleles in a single stranded form as a separate step in advance of heteroduplex formation is expensive and time consuming and the present invention aims to improve allele separation without the need for such separation by inducing the digestion of unwanted homo and heteroduplexes with endonucleases, e.g. restriction enzymes and optionally by using single stranded reference alleles in combination with double stranded sample alleles. Alternatively or additionally an enzyme specific for one of the strands of the sample and/or reference alleles, e.g. λ exonuclease can be used to generate single stranded forms of one or both of the sample and reference alleles and thus aid heteroduplex formation as described above. This method of inducing single strands of sample and/or reference alleles has the advantage that it can be carried out in situ in the reaction mixture and a separate step of single stranded preparation before the addition of the alleles to the heteroduplex forming mixture is not required.

Once heteroduplexes have been formed any suitable separating medium and apparatus can be used to separate the digested duplex molecules from the non-digested heteroduplex molecules. Appropriate media and means for allele separation are well known and documented in the art. Preferred separation means will allow the physical separation of molecules on the basis of one or more of size, conformation, hydrophobicity and charge and include polyacrylamide gel electrophoresis (PAGE), denaturing high performance liquid chromatography (DHPLC), capillary electrophoresis, mass spectrometry etc.

For DHPLC hydrophobicity of the DNA in the presence of particular buffers in addition to length of the molecule is of importance. The DNA is added to the column in the presence of a particular buffer (triethylammonium acetate (TEAA) buffer), which may bind the negatively charged phosphate groups of the DNA molecule on the one side and the hydrophobic matrix of the column on the other side. While increasing concentrations of hydrophobic buffer is run over the column, the DNA is eluted according to the speed with which the hydrophobisity of the buffer is increased since the buffer molecules will compete with the DNA in binding to the column matrix. Single stranded DNA expose more hydrogen atoms, thus heteroduplexed DNA will be more water soluble and less hydrophobic, and will therefore be eluted off the column earlier. On the same line of argument, single stranded and double stranded DNA may have different electrophoretic mobility due to differences in available charges. Thus, when DNA is separated by electrophoresis, heteroduplexes will tend to be less negatively charged compared to the homoduplexes of same molecular weight, and will therefore also for this reason be retarded in the gel.

As with all relatively labour intensive methods of analysis, automation is the ultimate goal. The ability to use DHPLC in conjunction with the improved methods of allele separation of the present invention allows for the automation of many of the steps of the method, including the step of automatic collection and monitoring of the peak positions. Thus, more preferred separation means are those which allow for some degree of automation and an especially preferred method of separation is DHPLC.

The methods of the present invention can be used to facilitate allele separation and hence genomic typing (i.e. the determination of the alleles present at a particular locus in a sample) of any genetic loci which contains one or more polymorphic positions. Thus, a further aspect of the invention provides a method of genotyping the alleles present in a biological sample, comprising subjecting said sample to the methods of allele separation as described herein and identifying the alleles present either by the migration pattern of the separated heteroduplexes and/or by direct sequencing of the sample alleles in the separated heteroduplexes. Appropriate methods of identifying the alleles present are described in more detail below. Generally, high resolution typing requires the determination of all or some of the nucleotide sequence of the alleles and thus methods involving sequencing steps are preferred. Such sequencing steps can be automated using methods well known and described in the art. Thus the methods of the invention will allow for fully automated sequence-based genotyping.

In particular the invention may be used for HLA typing (especially in relation to tissue typing, e.g. of bone marrow for transplantation), determination of polymorphisms involved in metabolism of pharmaceuticals, determination of mutations in disease loci, determination of mutations in cancers, determination of viral variants in chronic viral diseases, etc. In a preferred embodiment, the methods of allele separation described herein are used for HLA typing, and in particular HLA-A allele typing.

In a further embodiment, the invention can be used to diagnose particular diseases or susceptibility to diseases for which mutation information is known. Thus, yet further aspects of the invention include a method of diagnosis of disease in a subject or the susceptibility of a subject to a disease comprising subjecting a nucleic acid sample of said subject to a method of allele separation as defined herein and carrying out genomic typing to determine whether or not a particular mutation or mutations is (are) present.

The modifications/mutations to introduce and delete endonuclease sites, e.g. restriction enzyme sites to produce the modified reference alleles as described above can be carried out by any appropriate method and can conveniently be carried out by cloning the reference allele into an appropriate cloning vector and then carrying out site directed in vitro mutagenesis in conjunction with PCR. Such methods are well known and documented in the art and would be well within the bounds of a person skilled in the art.

Sites for one or more different restriction enzymes can be introduced and removed from the reference alleles, the only condition being that the sites are selected so that their location results in the digestion of sample allele homo or heteroduplexes or reference allele homoduplexes into fragments that are reduced in size compared to the full length heteroduplexes by an extent which is sufficient to allow separation of the digested fragments from the full length sample/reference allele heteroduplexes by an appropriate method of separation. Appropriate restriction enzymes to use can be selected by any convenient means. For example, a restriction enzyme map can be generated of the conserved regions of the particular genetic locus/allele in question and enzymes cutting at least once in appropriate positions in the conserved regions can easily be selected. Once selected all the restriction sites of this enzyme can then be deleted and new sites introduced as described above. As an illustration of this, the steps by which the Hae II restriction enzyme was selected for the HLA-A allele is described in Example 1.

In a preferred embodiment of the invention, as well as the reference alleles being modified/mutated to delete and/or introduce particular restriction sites, the reference alleles may be further modified at the sites which correspond to the sites in the sample alleles which bind the primers used for amplification of the sample alleles (sometimes referred to herein as the “sample primers”). Such mutations have been described for prior art methods (see e.g. Arguello et al., supra) and have the advantage of allowing the reamplification of the reference allele to be suppressed in the isolated heteroduplexes, thereby allowing the selective reamplification and optional subsequent manipulation and analysis (e.g. by sequencing) of the sample allele. Such sequencing analysis is generally required for the definitive allelic typing of the sample in question. This further modification of the reference alleles at the primer binding sites is also a preferred embodiment for the aspects of the invention where restriction enzymes are not used, i.e. in methods of allele separation involving the use of a reference allele and further comprising the use of double stranded sample alleles and/or the use of said reference allele in a double stranded form wherein one of the strands of the double stranded alleles present has been labelled so as to allow specific digestion of one of the strands.

The further modification of the reference alleles at the primer binding site can be carried out by any appropriate method and results in an alteration to the reference allele such that the primers which can bind to and are used to amplify the sample alleles can no longer bind appropriately to the equivalent region in the reference alleles, thereby suppressing re-amplification of the reference alleles using the sample primers. One convenient way to introduce such further modifications into the primer binding sites of the reference DNA is to use a set of mutated locus specific primers (sometimes referred to herein as “reference primers”) which are similar to the sample primers but which contain one or more mutations. Amplification of the reference alleles with such mutated reference primers will result in a primer mutation being incorporated into the amplified reference allele product. A particularly convenient way to design such mutated reference primers based on the sample primers is to extend the reference primers several bases (e.g. 2 to 10 bases, e.g. 3 to 8 bases, e.g. 5 to 6 bases) 3′ compared to the sample primers and, in addition, at the nucleotide position corresponding to the 3′ position of the sample primers introduce a nucleotide substitution.

Regardless of the way in which the modification in the primer binding site of the reference allele is introduced, when heteroduplexes between reference and sample DNA form and are isolated, the introduced modifications/mutations should not effect subsequent amplification and isolation of the sample allele by amplification with the sample primers but should prevent or significantly reduce amplification of the reference alleles, as the sample primers will no longer bind appropriately thereto.

In some instances it might be the case that a single mutation or modification introduced into the primer binding region of the reference allele does not sufficiently block the synthesis of the reference alleles with the sample primers. To further increase the specificity of the amplification, the sample primers may also be designed to contain a modification/mutation at a position some few bases from the 3′ end. This principle has been used extensively for development of primers for sequence specific PCR amplification of alleles (see for example Bottema C. D. et al., Mutat. Res., 1993, 288(1): 93-102). Such modifications still permit amplification of the sample alleles whereas the reference DNA will not be amplified due to several 3′ mismatches of the PCR primer.

In addition, or alternatively competitor primers may be added to the re-amplification mixture in order to further suppress the amplification of the reference strand.

Such competitor primers are thought to mask sites of non-specific primer-template interactions during amplification. However, for optimum amplification it is vital that the balance between specific and competitor primers is correct. If the specific primers are in excess, non-specific synthesis of DNA fragments prevails. On the other hand, a high excess of competitor primers may completely block sites of specific annealing in addition to the sites of non-specific annealing by competing out the specific primers. Thus, ideally an appropriate ratio between specific and competitor primers needs to be determined and this can easily be done by testing varying concentrations and ratios of the sample and competitor primers. Such determinations are routine when using competitor primers.

The introduction of the above-discussed further modifications/mutations at the primer binding sites of the reference alleles are however only preferred embodiments of the present invention as it is possible to carry out genotyping to some degree without the subsequent reamplification and analysis by sequencing of the sample alleles. In this regard, for any particular gene locus used in conjunction with one or more particular reference alleles, the heteroduplexes formed and the physical mobility thereof in the separation medium and separation conditions chosen will be slightly different for each particular heteroduplex and hence for each particular allele. Thus, it is possible to genotype the particular alleles present in a sample simply by monitoring and detecting the position/migration of the heteroduplexes under defined conditions and comparing these with known standards or other samples containing known alleles. Although it will be appreciated that this method is more crude than allele typing by sequencing it is also a lot faster and more convenient. Such “visual” typing based on the migration pattern of heteroduplexes will be particularly useful for the typing of loci which have only a few allelic variations and where each of these variants forms a heteroduplex with a reference allele which has a migration band or peak that is sufficiently different from all the other possible heteroduplexes. In addition, such “visual” typing is also very useful for comparing samples for tissue matching purposes.

The monitoring/detection of the migration bands or peaks can be carried out by any appropriate method. For example, the nucleic acid in the heteroduplex bands can be stained using ethidium bromide and visualized under U.V. light, or can be visualised under an appropriate fluorescent light source if the reference or sample alleles used have been labelled with a fluorescent tag. (The use of a fluorescent reference allele and the analysis of fluorescent duplexes are described Arguello et al, 1998, Tissue Antigens 52:57-66). Alternatively, if column separation is used the migration and/or elution position of the peaks of the various duplexes under defined conditions can be automatically detected by the column equipment and compared with known standards or known samples. The monitoring and detection of peaks in this way would be routine to a person skilled in the art.

Once the modified reference allele or reference allele construct has been designed it can be used as a template for PCR to produce enough amplified copies for use in one or several assays. Depending on the design of the modified reference allele (i.e. whether or not the reference allele has been subject to further modification in the primer binding region), the primers for amplification of the reference allele (the reference primers) may be the same or different from the primers used to amplify the sample alleles (the sample primers). Generally the modified reference allele construct will be amplified separately from the sample alleles and then mixed with the amplified sample alleles in a subsequent step.

In the embodiments where it is desired to use only one of the strands of the reference allele in a particular experiment the desired strand can be separated by methods well known and documented in the art. For example, this can be achieved by the biotinylation of one of the reference primers (i.e. either the forward or reverse reference primer), thereby resulting in the strands amplified by the extension of such primers also have a biotin label. The labelled strands (which correspond to only one of the two complementary strands of the reference allele) can then be separated from the non-labelled strands by for example binding them to streptavidin coated magnetic beads (or other solid supports) and then separating the bound strands from the non-bound strands using a magnetic field. Once separated both the strands of the reference alleles can be used in separate experiments.

As described above methods whereby the separation of both the reference and the sample alleles is required for allele separation are known in the art. It will be appreciated however that the preparation of only the reference DNA in a single stranded form rather than having to separate the two strands of both alleles of the sample DNA, clearly simplifies the preparation of the alleles before the separation procedure and is advantageous over the prior art methods in terms of cost and labour savings. In addition, the single stranded reference DNA can be prepared in batches beforehand and the quality of the preparation could be checked before it is used for heteroduplex induction in the allele separation methods as described herein.

Alternatively, in the methods of the invention where an enzyme resulting in specific digestion of one of the strands (e.g. λ exonuclease) of double stranded alleles is used, the preparation of either or both the reference and/or sample alleles in a single stranded form can be carried out in situ in the reaction medium. This avoids the problems of the prior art methods where separation of the individual strands of both the sample and reference alleles is required before they are added to the reaction mixture in which the heteroduplexes are allowed to form. In such methods appropriate unwanted strands of the sample and/or reference alleles are labelled such that digestion of one of the strands is specifically induced. For example, in the case of λ exonuclease the unwanted strand is labelled with a 5′phosphorylated group and this strand is then specifically digested by λ exonuclease leaving single stranded sample and/or reference alleles which can then, under appropriate conditions, undergo heteroduplex formation. The use of enzymes such as λ exonuclease are therefore alternative methods of single stranded allele formation and also alternative or additional methods of aiding heteroduplex formation. Appropriate 5′ end labelling of the appropriate strands of the sample and/or reference alleles can be readily induced by for example using an appropriate 5′ end labelled primer for amplification.

The sample containing the particular alleles to be analysed may be any sample containing genetic material and can be derived from any appropriate source. All biological and clinical samples are included, i.e. any cell or tissue sample of an organism, or any body fluid or preparation derived therefrom, as well as samples such as cell cultures, cell preparations, cell lysates etc. The samples may be freshly prepared or they may be prior-treated in any convenient way e.g. for storage. Thus, the sample will generally be a biological sample, which may contain any viral or cellular material, including all prokaryotic or eukaryotic cells, viruses, bacteriophages, mycoplasms, protoplasts and sub-cellular components such as organelles. Such biological material may thus comprise all types of mammalian and non-mammalian animal cells, plant cells, insect cells, algae including blue-green algae, fungi, bacteria, protozoa etc. Preferably the samples will be derived from human or animal sources including rat, mouse, goat, sheep, rabbit, dog, monkey, etc. Representative samples thus include whole blood and blood-derived products such as plasma, serum and buffy coat, urine, faeces, cerebrospinal fluid or any other body fluids, tissue samples, organ samples, cell cultures, cell suspensions etc., derived from the afore-mentioned human or animal sources.

Before the sample can be assayed in accordance with the present invention, nucleic acid and preferably DNA should be extracted from the source. This can be done by methods well known and documented in the art and appropriate methods would be known to a skilled man depending on the nature of the sample obtained. In general, where the methods of the present invention are used to analyse DNA alleles it is not necessary to separate the DNA from other nucleic acids which may be present (e.g. RNA). Thus, a relatively crude nucleic acid containing preparation can be used, for example a lysate of cells, digested with proteinase K. The main requirement is that the nucleic acid preparation must be sufficiently free of impurities to enable amplification of the sample alleles to be carried out, e.g. be free of any substances which might inhibit or degrade the enzymes involved in PCR. In addition, of course, enzymes which degrade DNA should be absent.

Once the nucleic acid has been extracted from the sample, the sample alleles/gene loci of interest, or fragments thereof, are generally amplified by PCR using appropriately designed locus specific sample primers, i.e. primers which are designed to flank the allele/loci or fragment of interest and allow amplification, under appropriate conditions, of the DNA in between said flanking primers. The selection of appropriate PCR conditions and primers would be well within the bounds of a person skilled in the art.

The PCR amplified sample is then mixed with an appropriate amount of either one or both strands (depending on the design of the experiment) of the appropriate modified reference alleles or reference alleles. The sample alleles and reference alleles are mixed under appropriate conditions to enable heteroduplexes to form. Again the selection of appropriate conditions would be obvious to a skilled man. Exemplary conditions include the heating of the sample to a temperature and for a time sufficient to denature the double stranded nucleic acid present (e.g. heating at 95° C. for 5 minutes), chilling on ice and then allowing the nucleic acid strands to reanneal/hybridise at 65° C. for approximately 30 minutes before chilling the samples again on ice.

Once heteroduplexes (and homoduplexes) have formed, the selected endonucleases, e.g. restriction enzymes are added (in the embodiments of the invention where endonucleases, e.g. restriction enzymes are used), again under appropriate conditions (e.g. time and temperature, ion concentration etc.) to allow digestion to occur. The particular conditions which are appropriate for any restriction enzyme are well known in the art and generally provided in the manufacturer's instructions which accompany the commercially bought enzyme. For example, in the case of Hae II, which is a preferred enzyme for use in the separation of HLA-A alleles, exemplary conditions are the use of 10 units of enzyme at 37° C. for one hour. As described above, under the appropriate conditions, digestion of homoduplexes of reference alleles and homo and heteroduplexes of sample molecules will be induced, whereas the heteroduplexes of sample and reference alleles will be protected from digestion. In other words, all molecules not formed as a heteroduplex with the reference allele are shortened by digestion with the selected restriction enzyme(s).

In embodiments of the invention where enzymes such as λ exonuclease are used alternatively or in addition to endonucleases, e.g. restriction enzymes to aid heteroduplex formation, such enzymes are added at the appropriate time and under appropriate conditions to allow digestion to occur. Such enzymes may be added before, at or around the same time and optionally under the same conditions as the restriction enzymes. Preferably however the enzymes such as λ exonuclease are added before the restriction enzymes and before the induction of heteroduplexes (e.g. after the sample and reference alleles have been mixed) under appropriate conditions to allow the enzyme to act.

Once formed, the heteroduplexes are then separated from the digested molecules in the mixture by any suitable separation means as described above.

Depending on the separation means used, the analysis/typing of the alleles present in the sample may be carried out directly by analysing the migration positions of the bands or peaks which correspond to the heteroduplexes present. Alternatively the bands or fractions containing the separated heteroduplexes of the sample can be isolated or collected and the alleles contained therein be identified by sequencing of the sample allele. If the sample alleles are present as a band within a gel, the band is excised and the nucleic acid extracted and analysed (e.g. sequenced) by any of the methods well known and described in the art. If the sample alleles to be sequenced have been obtained from a column then generally the sequencing can be carried out directly on the isolated fraction. In general to remove interference from the unwanted reference allele during the sequence analysis a reamplification step as described above, which is specific for the sample allele, is usually carried out before sequencing takes place. Providing the modified reference allele construct has been designed to contain an extra mutation or mutations (or other modifications) in the primer binding region as described above, this specific reamplification is easily effected by using the same sample primers as were used to amplify the original sample DNA.

A further aspect of the present invention thus provides an improved method of separation of alleles in a sample, comprising the use of a modified reference allele, wherein said modified reference allele comprises one or more modifications (e.g. mutations) such that heteroduplexes formed between said reference allele and an allele present in the sample (sample allele) will be resistant to digestion with one or more endonucleases, e.g. restriction enzymes which will digest homoduplexes or heteroduplexes of said sample alleles or homoduplexes of said reference alleles, wherein said method comprises the steps of
(i) mixing said reference alleles with said sample alleles under conditions such that heteroduplexes are formed,
(ii) addition of an appropriate endonuclease(s), e.g. restriction enzyme or enzymes to digest all duplexes except the heteroduplexes formed between the sample and the reference alleles,
(iii) separation of the digested and non-digested fragments and, optionally,
(iv) analysis of the separated fragments.

In further embodiments of the invention as described above one of the strands of one or both of the sample and reference alleles may be appropriately labelled so as to allow specific digestion of one of the strands using an enzyme. In such embodiments where λ exonuclease is used to digest the labelled strands, one of the strands of one or both of the sample and reference alleles are labelled with 5′ phosphorylated label and λ exonuclease is added in step (ii) at or around the same time as the restriction enzymes. Alternatively where enzymes such as λ exonuclease are used these may be added before heteroduplex induction, i.e. step (i) above may comprise mixing said reference alleles with said sample alleles, addition of an appropriate enzyme (e.g. λ exonuclease) which specifically digests one of the strands of appropriately labelled double stranded reference and/or sample alleles and incubation of the mixture under conditions such that heteroduplexes are formed.

An alternative aspect of the present invention provides an improved method of separation of alleles in a sample (sample alleles), involving the use of a reference allele and further comprising the use of double stranded sample alleles and/or the use of said reference allele in a double stranded form wherein one of the strands of the double stranded alleles present has been labelled so as to allow specific digestion of one of the strands, wherein said method comprises the steps of:
(i) mixing said reference alleles with said sample alleles;
(ii) addition of an appropriate enzyme which specifically digests one of the strands of the appropriately labelled double stranded reference and/or sample alleles;
(iii) subjecting the mixture to conditions such that heteroduplexes are formed;
(iv) separation of the digested and non-digested fragments and, optionally
(v) analysis of the separated fragments.

Alternatively the enzyme, rather than being added in step (ii) of this method, may be added during or after step (iii), i.e. during or after heteroduplex formation. A preferred enzyme for use in these methods is λ exonuclease which specifically digests strands of nucleic acid containing a 5′ phosphorylated label.

Preferred reagents for use in such methods and more detail as to how the individual steps are carried out are provided above.

Although modified reference alleles which contain mutations within the sample primer binding regions are known in the art, reference alleles which have been modified so as to delete or inactivate certain endonuclease sites, preferably restriction enzyme sites which sites are present in the sample alleles, and introduce alternative endonuclease sites, preferably restriction enzyme sites for the same enzyme or enzymes are not described. Such modified reference alleles and their use in methods of allele separation and in heteroduplex formation form yet further aspects of the invention. Vectors comprising such modified reference alleles are also included in the scope of the present invention.

The present invention also provides methods for preparing such modified reference alleles, said methods comprising the steps of (i) selecting a sample allele, (ii) identifying one or more endonuclease enzymes, preferably restriction enzymes which cleave all known sample alleles at least once, (iii) deleting or inactivating the endonuclease sites, preferably restriction sites for these enzymes and (iv) introducing one or more alternative sites for the same enzyme into the allele to form the modified reference allele.

Modified reference alleles obtainable by the above described method are also included as are vectors comprising said modified reference alleles.

Preferred modified reference alleles of the invention comprise further modifications/mutations in the sample primer binding site as described above, such that the sample primers can no longer anneal appropriately to the reference allele and the reference allele can no longer be amplified efficiently by use of the sample primers. More preferred modified reference alleles are modified HLA alleles, especially preferably modified HLA-A alleles. A particularly preferred modified reference allele for use in HLA-A analysis is as defined in SEQ ID NO: 1.

Thus, a further aspect of the present invention provides nucleic acid molecules comprising a nucleotide sequence as defined in SEQ ID NO. 1, or a fragment thereof comprising a functionally active sequence, or a sequence which is degenerate, substantially homologous with or which hybridises with the sequence as defined in SEQ ID NO. 1 or with the sequence complementary thereto, or a fragment thereof encoding a functionally active product.

“Functionally active sequence” as used herein refers to any modified reference allele as defined above which is capable of forming a heteroduplex with one or more of the sample alleles for a particular gene and which heteroduplex has a physical conformation different from a homoduplex of the sample alleles or a heteroduplex of the sample alleles or a homoduplex of the reference allele such that the heteroduplexes formed between the sample and reference alleles can be physically separated from any homoduplexes formed and also any sample allele heteroduplexes in an appropriate separation medium. In addition such modified reference alleles have been modified so as to delete certain restriction enzyme sites which sites are present in the sample alleles, and to introduce alternative restriction sites for the same enzyme or enzymes.

“Substantially homologous” as used herein includes those sequences having a sequence homology (or a sequence identity) of approximately 60% or more, e.g. 70%, 75%, 80% or 85% or more and also functionally equivalent allelic variants and related sequences modified by single or multiple base substitution, addition and/or deletion. By “functionally equivalent” in this sense is meant “functionally active sequences” as defined above.

For determining the degree of homology (or identity) between sequences, computer programs that make multiple alignments of sequences are useful, for instance Clustal W (Thompson, J. D., D. G. Higgins, et al. (1994). “CLUSTAL W: Improving the sensitivity of progressive multiple sequence alignment through sequence weighting, position-specific gap penalties and weight matrix choice”. Nucleic Acids Res 22: 4673-4680). One especially useful feature of this program is that, in cases where structure information is available for one or more members of the alignment, it can be set to use such information to improve the quality of the alignment. Programs that compare and align pairs of sequences, like ALIGN (E. Myers and W. Miller, “Optical Alignments in Linear Space”, CABIOS (1988) 4: 11-17), FASTA (W. R. Pearson and D. J. Lipman (1988), “Improved tools for biological sequence analysis”, PNAS 85:2444-2448, and W. R. Pearson (1990) “Rapid and sensitive sequence comparison with FASTP and FASTA” Methods in Enzymology 183:63-98) and gapped BLAST (Altschul, S. F., T. L. Madden, et al. (1997). “Gapped BLAST and PSI-BLAST: a new generation of protein database search programs”. Nucleic Acids Res. 25: 3389-3402) are also useful for this purpose. Furthermore, the Dali server at the European Bioinformatics institute offers structure-based alignments of protein sequences (Holm, J. of Mol. Biology, 1993, Vol. 233: 123-38; Holm, Trends in Biochemical Sciences, 1995, Vol 20: 478-480; Holm, Nucleic Acid Research, 1998, Vol. 26: 316-9).

By way of providing a reference point, sequences according to the present invention having 60%, 70%, 75%, 80%, 85% homology (identity) etc. may be determined using the ALIGN program with default parameter for instance available on Internet at the GENESTREAM network server, IGH, Montpellier, France).

Sequences which “hybridise” are those sequences binding (hybridising) under non-stringent conditions (e.g. 6×SSC, 50% formamide at room temperature) and washed under conditions of low stringency (e.g. 2×SSC, room temperature, more preferably 2×SSC, 42° C.) or conditions of higher stringency (e.g. 2×SSC, 65° C.) (where SSC=0.15M NaCl, 0.015M sodium citrate, pH 7.2).

Generally speaking, sequences which hybridise under conditions of high stringency are included within the scope of the invention, as are sequences which, but for the degeneracy of the code, would hybridise under high stringency conditions.

Kits for allele typing which comprise such modified reference alleles form a yet further aspect of the invention. Other optional components of the kits include sample primers to amplify the particular sample alleles concerned, reference primers to amplify the modified reference allele, appropriate restriction enzymes (or other appropriate endonucleases) and optionally appropriate exonucleases to digest the irrelevant duplexes formed and buffers to promote heteroduplex formation. Preferred kits have as optional components one or more appropriate restriction enzymes, e.g. HaeII and/or λ exonuclease.

Preferred kits are suitable for the typing of HLA alleles, preferably HLA-A alleles and the modified reference alleles supplied in the kits are HLA alleles, preferably HLA-A alleles which have been modified as described above. An especially preferred HLA-A modified reference allele is defined in SEQ ID NO. 1.

An alternative aspect of the invention provides a method of separation of alleles in a sample, wherein the alleles are separated by denaturing high-performance liquid chromatography (DHPLC), comprising the use of a modified reference allele which can form heteroduplexes with alleles contained within the sample (sample alleles) and which contains a plurality of additional modifications/mutations such that improved separation of alleles is obtained.

“modified reference alleles” as used herein in connection with this alternative aspect of the invention has a meaning essentially as defined above, i.e. these “modified reference alleles” essentially have all the properties of the reference alleles as defined above except for the fact that they do not correspond to alleles found in vivo, by virtue of the plurality of additional modifications/mutations which are present. Thus in developing a modified reference allele for use in this aspect, generally a reference allele is taken (which corresponds to a sample allele, most probably a rare sample allele, or which corresponds to the wild type allele, if appropriate) and additional mutations/alterations are generated. These additional mutations/alterations which have the effect of leading to improved separation of the sample alleles can be at any location in the reference allele and can be of any type which can induce mismatch between a sample and reference allele, such as those discussed above. The mutations/modification can lead to the introduction or deletion of a restriction site (i.e. can be modifications/mutations such as those described above for the first aspect of the invention) or can be simple mismatch mutations which do not introduce or delete such restriction sites. Thus, such additional modifications/mutations are not necessarily located at any particular position in the modified reference allele and are generally spread throughout the length of the reference allele. Any number of additional mutations/alterations may be generated provided that these alterations result in an improved allele separation. Preferably at least 1%, for example between 1% and 10% or even up to 20% or up to 30% of the bases, more preferably between 1% and 5% and most preferably approximately 1% of the bases of the reference allele are modified/mutated to generate the “additional modifications/mutations”.

Viewed another way, preferred modified reference alleles are designed so that at least 1%, for example between 1% and 10%, or even up to 20% or up to 30% of the total bases making up the modified reference allele form mismatches with the various sample alleles when heteroduplexes are formed. These mismatches may be generated by the introduction of mutated or otherwise modified bases into the reference allele and/or may be generated automatically due to inherent sequence differences between the particular sample alleles in the sample and the modified/mutated reference allele selected. Thus, the number of additional modifications/mutations that need to be introduced into the reference allele to effect improved separation may vary depending on the number of inherent mismatches between the reference allele and the sample alleles. Generally up to 10, 20 or 30 additional modifications/mutations may be introduced or at least 1%, e.g. between 1% and 10% or even up to 20% or up to 30% of the bases of the reference allele may be modified/mutated as described above.

It is expected that there is an optimum percentage of mutations/mismatches which gives the best separation for a particular allele and that a number of modifications/mutations above or below this value will give less efficient separation (see FIG. 2a). Thus, the above percentage values are only approximations and it would be well within the bounds of a skilled man to eludicate the number of mutations/mismatches which can result in the improved separation in accordance with this aspect of the invention.

“improved separation” as used herein is necessarily a relative term and refers to heteroduplexes between the sample and modified reference alleles being further separated in the chosen separation medium than the separation observed when a reference allele containing no additional modifications/mutations (i.e. which corresponds to the wild type gene or a sample allele, as appropriate, depending on the genetic locus concerned) or fewer additional modifications/mutations than the modified reference allele in question is used to form heteroduplexes. “improved separation” thus refers to a measurably better separation of the alleles when compared to the separation observed when a reference allele containing no additional modifications/mutations or fewer additional modifications/mutations than the particular modified reference allele in question is used. Whether or not such separation is measurably improved will of course depend on the sensitivity of the method used to detect separation. Preferably the improvement in separation will be statistically significant. In DHPLC such improved separation corresponds to heteroduplex peaks/fractions being measurably further apart (and preferably statistically significantly further apart). Examples of such “improved separation” of alleles can be seen in FIGS. 2b and 2c.

Such improved separation (i.e. the observation that the DHPLC peaks are further apart) observed when a plurality of additional modifications/mutations are introduced is especially useful for allele separation, where often several alleles tend to produce heteroduplexes with the same migratory/elution profile when reference alleles which correspond to wild type or sample alleles are used. Thus, the inclusion of one or several additional modifications/mutations increases the likelihood that these different alleles will migrate/elute differentially.

DHPLC provides a highly sensitive and automated method to resolve homoduplex from heteroduplex molecules under conditions of partial heat denaturation within a linear acetonitrile gradient (see e.g. Underhill et al., 1997, Genome Research, 7:996-1005 and Underhill et al., 1996, PNAS USA 93:196-200). Heteroduplex molecules generally have shorter retention times than homoduplexes and under the correct conditions can be visualised as distinct peaks and eluted from the column in distinct fractions. The degree of separation of the homo and heteroduplexes is quite dependent on temperature and it is standard procedure to test a range of temperatures (e.g. between 50 and 65° C.) in order to find an optimum temperature for separation. A difference in retention times of the different homo and heteroduplexes of at least a minute is ideal. Alternatively, the WAVE™ DNA Fragment Analysis System (Transgenomic, Inc, San Jose, Calif.) machine can be used to predict the melting temperature of a particular heteroduplex sequence. Also, algorithms exist which predict the melting temperatures (http://insertion.stanford.edu/melt.html) based on the sequence composition of a fragment.

Once the appropriate conditions have been elucidated the characteristic elution profiles and shapes of the DNA peaks can be used to genotype unknown samples independently of sequence analysis by comparison with known sample profiles. If it is desired to sequence particular heteroduplexes then this can generally be carried out immediately (usually after the desired sequences have been amplified, e.g. by PCR, as described above) on the collected fraction concerned, without the need for pre-treatment.

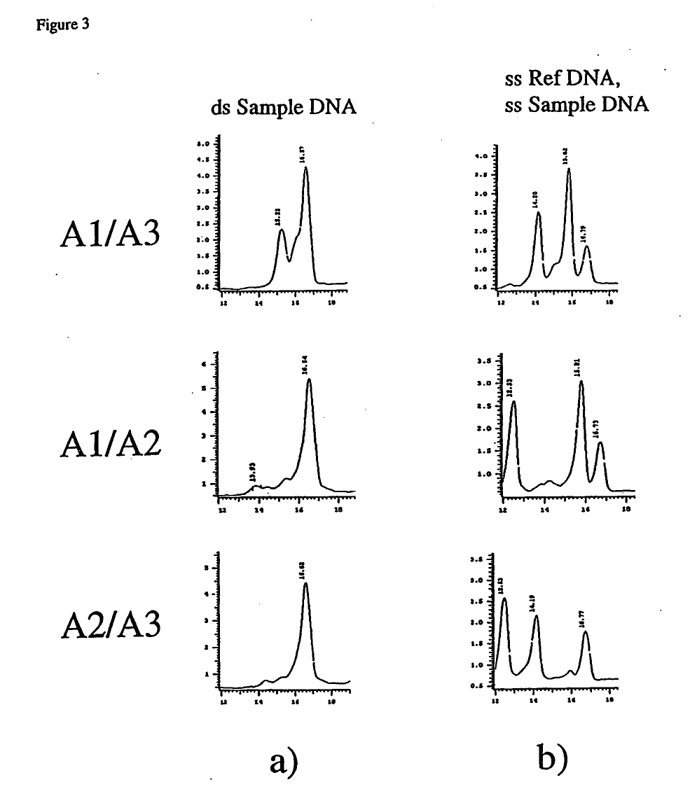

Whilst not wishing to be bound by theory it is believed that the improved separation caused by the plurality of additional modifications/mutations in the reference allele is that the heteroduplexed molecules which are formed are more open in structure, i.e. consist of several regions of single stranded DNA. In DHPLC double stranded DNA binds well to the column, and it is only eluted off the column when the concentration of the hydrophobic elution buffer reaches a critical level. The concentration of elution medium and temperature at which elution occurs is highly dependent on the length of the DNA, and elution profiles of DNA of given length is highly reproducible. However, if the DNA contains single stranded stretches (i.e. heteroduplexes), DNA of the same length will elute off the column more rapidly, that is at a lower concentration of elution buffer. If the DNA binds firmly to the column it will not be possible to elute off the DNA at conditions which also allow for detection of heteroduplexed DNA. Thus it is generally assumed that DHPLC may be used for reliable discrimination of DNA molecules less than 500 bp length. However, by using a modified reference allele containing a plurality of additional modifications/mutations in accordance with this aspect of the invention, this length limit may be exceeded so that fragments of approximately and at least 1000 bp in length can be separated (see e.g. FIGS. 3a and 3b). This is clearly an important advantage.

Thus, the use of such modified/mutated reference alleles has the advantage that larger fragments (which in previous methods would have required too high a concentration of elution buffer to elute and detect heteroduplexes) can be separated and also that fragments which were previously thought to be indistinguishable by DHPLC may be distinguishable. In this regard, as discussed above, the Wave instrument has a software which analyses the melting profile of fragments, which is used as an indication as to whether it will or will not be possible to distinguish sequence differences between two DNAs in the same sample (i.e. heteroduplexes). However, we believe that by introducing additional modifications/mutations in the reference DNA in accordance with the present invention, in fragments that are predicted not to allow for identification of sequence differences, heteroduplexes that can be distinguished will be obtained.

The invention will now be described in more detail in the following non-limiting Examples with reference to the following drawings in which:

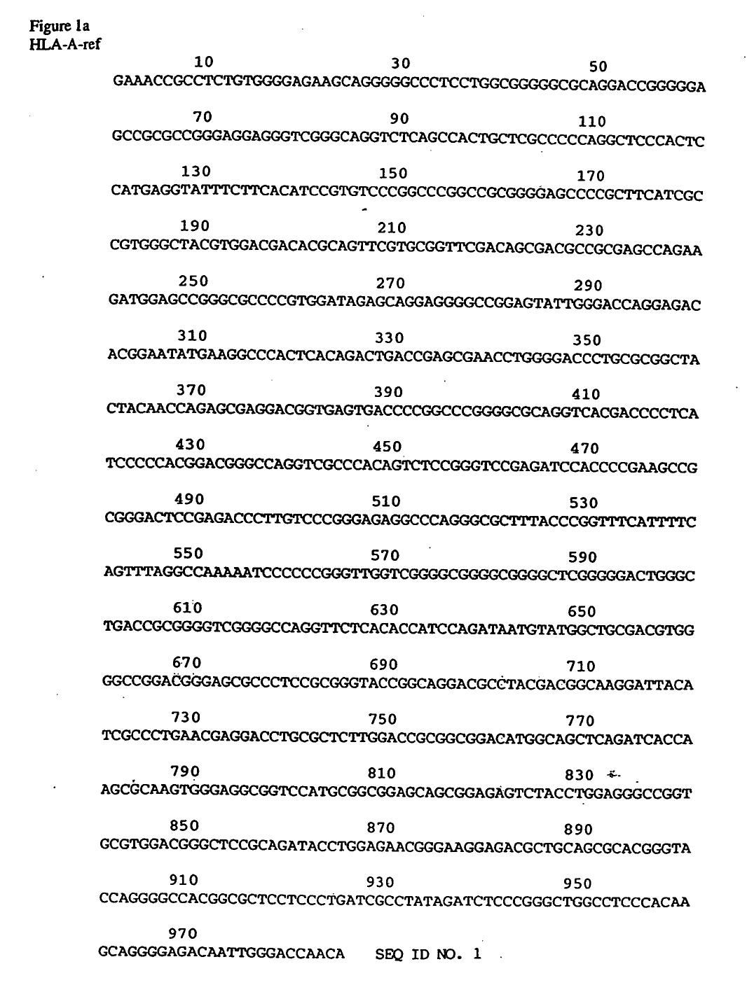

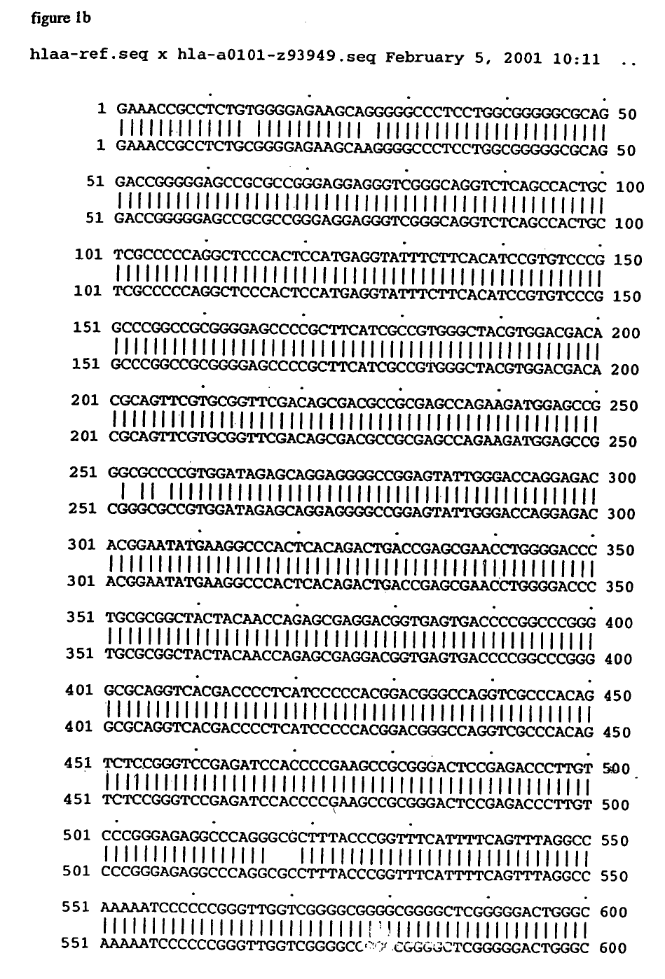

FIG. 1: FIG. 1a shows the sequence of the mutated HLA-A*0101 reference probe (SEQ ID NO. 1). The sequence includes the mutated HaeII recognition sites, as well as the mutations in the primer sites. The sequence is derived from z93949. In panel b, a comparison of A*0101 and the mutated reference probe is shown, with identities between the sequences marked with |.

FIG. 2: FIG. 2a shows a theoretical representation of the utility of introducing mutation in the reference probe. There exists probably an optimal number of mutations, maximizing the separation distance between both alleles and the homoduplexes. Few mutations yields molecules which are eluted in a similar way to the homoduplex (marked homo), and with not much difference in elution time between the allele A1 and A2. More mutations increases the distance both between A1 and A2, and between each of the alleles with the homoduplex peak. Even more mutations shortens the elution time even more, and the distance to the homoduplexes increases further, but the distance between the two alleles are again shorter.

FIG. 2b shows results showing increasing separation distance between homoduplex and heteroduplexes with increasing number of mutated regions in the HLA-A ref probe. Heteroduplexes were generated using single stranded sample and reference DNA. Ref-250 is the HLA-A*0101 allele amplified with HLA-AF-ref and HLA-AR-ref primers and with the HaeII mutations depicted in FIG. 1b introduced at position 250. Similarly Ref-250/500 is the same as Ref-250 with HaeII mutations also introduced at position 500 etc. A schematic representation of the location of the mutated regions in the reference probe is given. N refers to non-sense strand. The diagrams to the right show DHPLC separation of heteroduplexes using reference DNA with increasing number of mutated regions from a homozygous HLA-A1 sample and a heterozygous HLA-A1/A3 sample.

FIG. 2c is a diagram showing relative increase in separation distances observed in the experiment shown in FIG. 2b. Both separation distance of heteroduplex from homoduplex, and separation distance between heteroduplexes increases with increasing number of mutated regions in the reference probe.

FIG. 3 shows the separation of HLA-A alleles using DHPLC and mutated reference probe. PCR amplification conditions and primers used are described in the examples section. Where single stranded (ss) DNA was used, this was obtained using PCR products where either the sense or the antisense strand was biotinylated. Removal of the biotinylated strand was performed using streptavidin M280 Dynabeads. Heteroduplexes where induced as described in the examples section. After induction of heteroduplexes, the samples were injected one by one into the column of the DHPLC apparatus. The diagram shows intensity of peaks eluted from the column as measured by UV light. In panel a, it is shown that heteroduplexes in HLA-A heterozygous samples is not always observed. Double stranded (ds) PCR products of three different heterozygous samples are shown. No reference probe is added, and it can be seen that in the A*0101/A*0201 heterozygous (A1/A2) and the A*0201/A*0301 heterozygous (A2/A3) samples no heteroduplexes may be discerned. In the A*0101/A*0301 heterozygous sample (A1/A3) however, a heteroduplex peak may be seen. In panel b, single stranded sense sample DNA is mixed with single stranded antisense reference DNA. Heteroduplexes are formed, and migrate in a reproducible manner according to the alleles present in the sample. Homoduplexes are also seen, indicating that the purification of the ss DNA, using biotinylated PCR products and magnetic beads, was not complete.

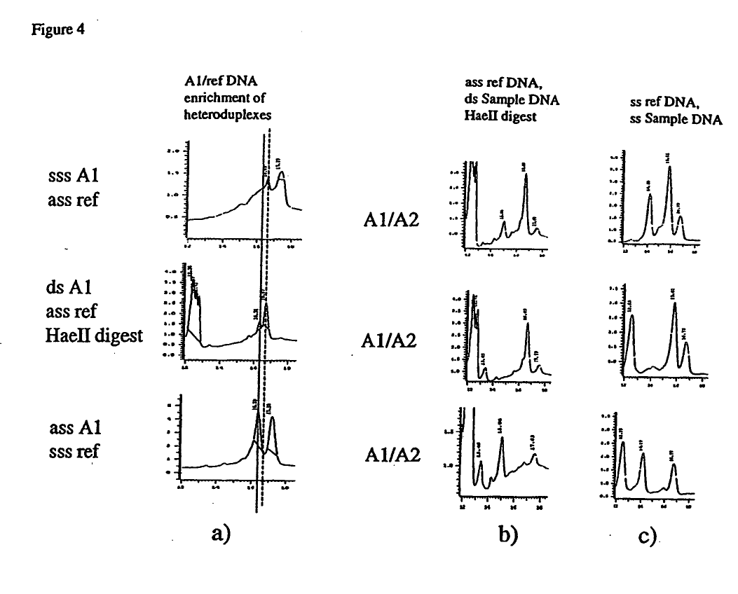

FIG. 4 shows the enrichment of heteroduplexes by HaeII digestion as visualised with DHPLC separation using the Wave equipment. The experiment is carried out as described for FIG. 3. Panel a shows the enrichment of heteroduplexes of A1*0101 by HaeII digestion. ds is double stranded DNA, sss is sense single stranded DNA, and ass is antisense single stranded DNA. In this panel it can also be clearly seen that the reference probe is not purely single stranded, since the heteroduplex generated between ass A1 and sss ref DNA (marked with the left stipled line) is visible also in the panel with digested ds A1 with ass ref DNA where the heteroduplex between sss A1 and ass ref DNA is dominating (marked with the right stipled line). In panel b the same three heterozygous combinations from FIG. 3 are shown after heteroduplex induction and HaeII digestion for enrichment of heteroduplexes. The major peaks are digested DNA, the heteroduplex peaks are rather weak, indicating that the induction of heteroduplexes in this experiment was not optimal. Panel c is the same as panel b in FIG. 3, and is included to allow direct comparison of peak migration.

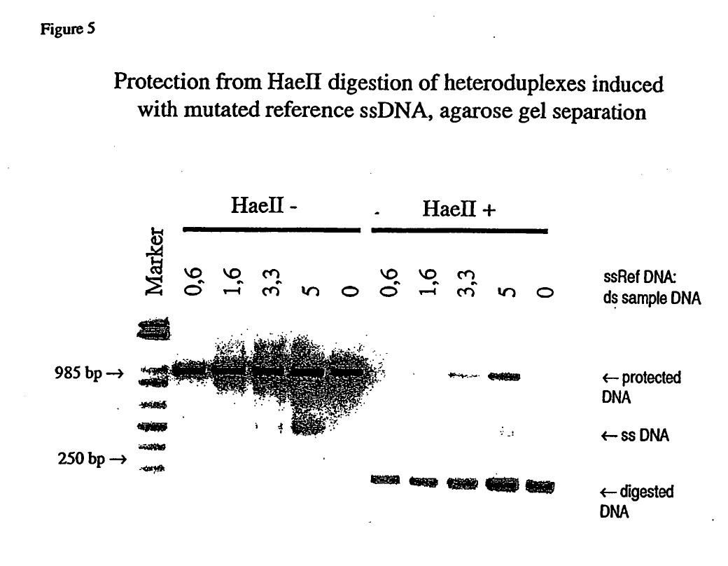

FIG. 5 shows the inhibition of digestion of heteroduplexes by using the mutated HLA-A*0101 reference probe as defined in SEQ ID NO. 1. Single stranded reference DNA was prepared and added to double stranded sample DNA in increasing amounts. The numbers above the lanes refer to the relative amount of single stranded reference DNA in relation to the fixed amount of double stranded sample DNA used in all 10 lanes. Heteroduplexes were allowed to form and the samples digested (lanes 6 to 10) or not digested (lines 1 to 5) with HaeII (as shown in the figure). DNA was visualised with ethidium bromide staining after agarose electrophoresis. The lane marked ‘Marker’ contains molecular weight markers.

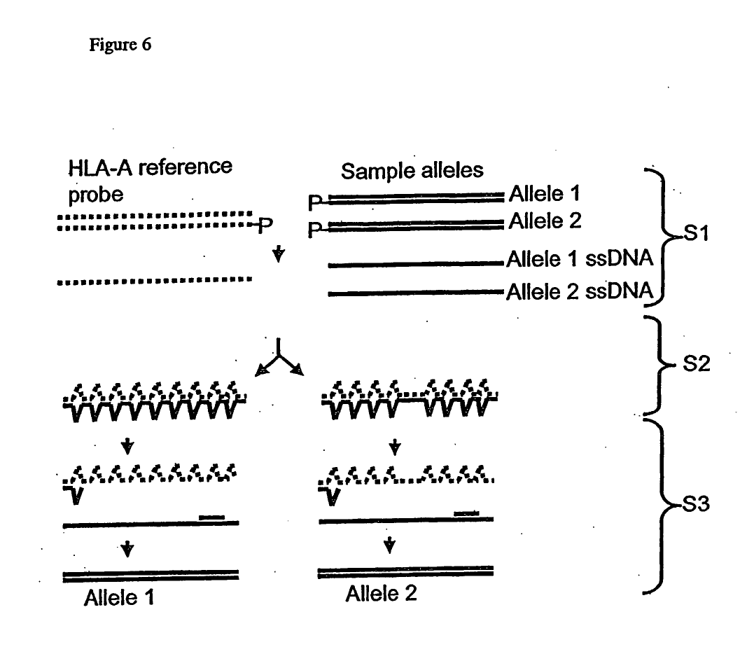

FIG. 6. Schematic presentation of the method of DHPLC-SBT of polymorphic gene loci alleles. S1 depicts isolation of complementary strands of the reference and sample DNA by λ-exonuclease. S2 shows the induction of heteroduplexes between the isolated ssDNA and subsequent separation by DHPLC. S3 displays the reamplification of the sample alleles alone from separated heteroduplexes before sequencing.

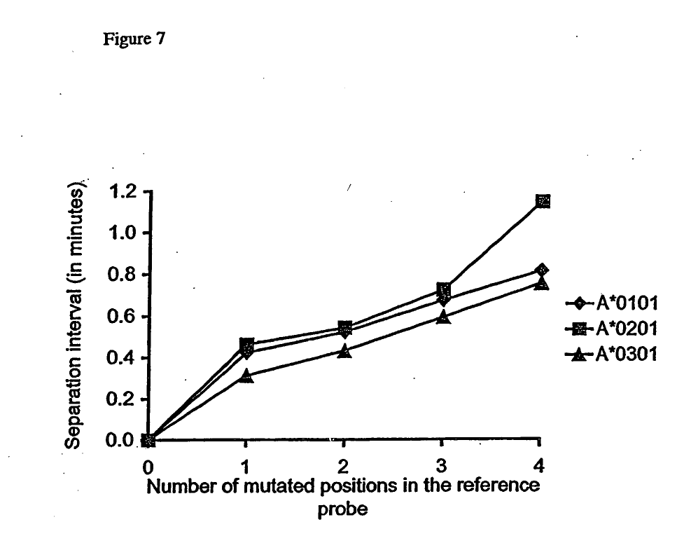

FIG. 7. HLA-A reference probe constructs facilitate HLA-A allele separation by DHPLC. Four reference constructs having mutations at 1, 2, 3 and 4 conserved regions respectively in HLA-A exon 2 and 3 fragment were applied.

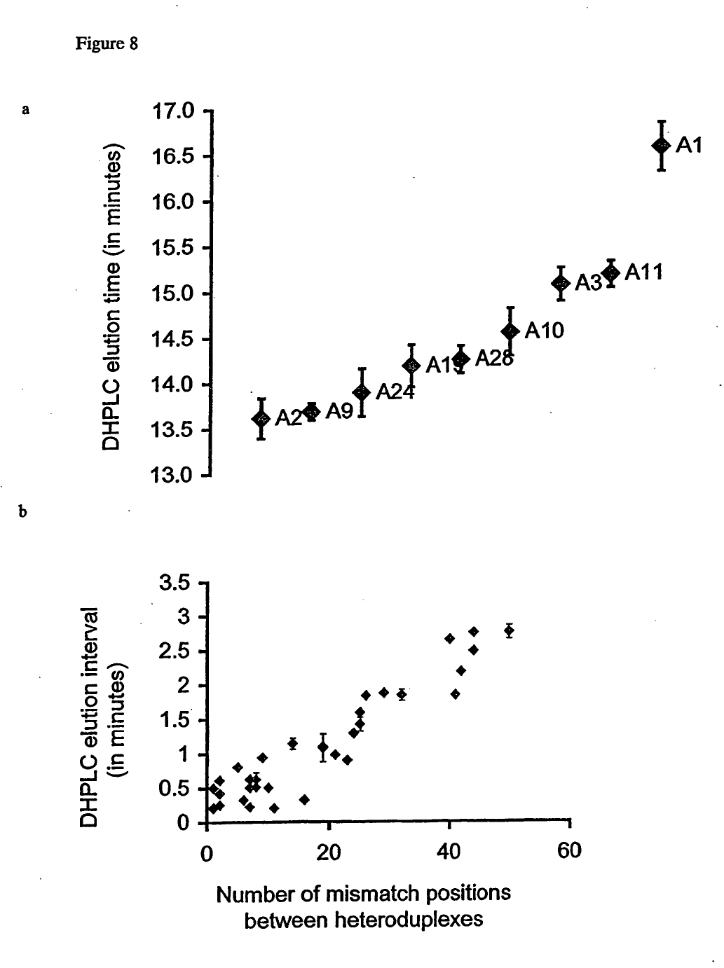

FIG. 8. Influence of TOPO-A*0101-mut13 reference probe on the separation of HLA-A alleles. Panel a depicts the specific elution profile of HLA-A generic groups as resolved by DHPLC. Panel b displays the resolution of HLA-A heterozygous samples by DHPLC. Error bars show the standard deviation (SD) of the HLA-A generic subgroups DHPLC elution time (panel a) and the SD for the DHPLC separation interval of HLA-A heterozygotes (panel b).

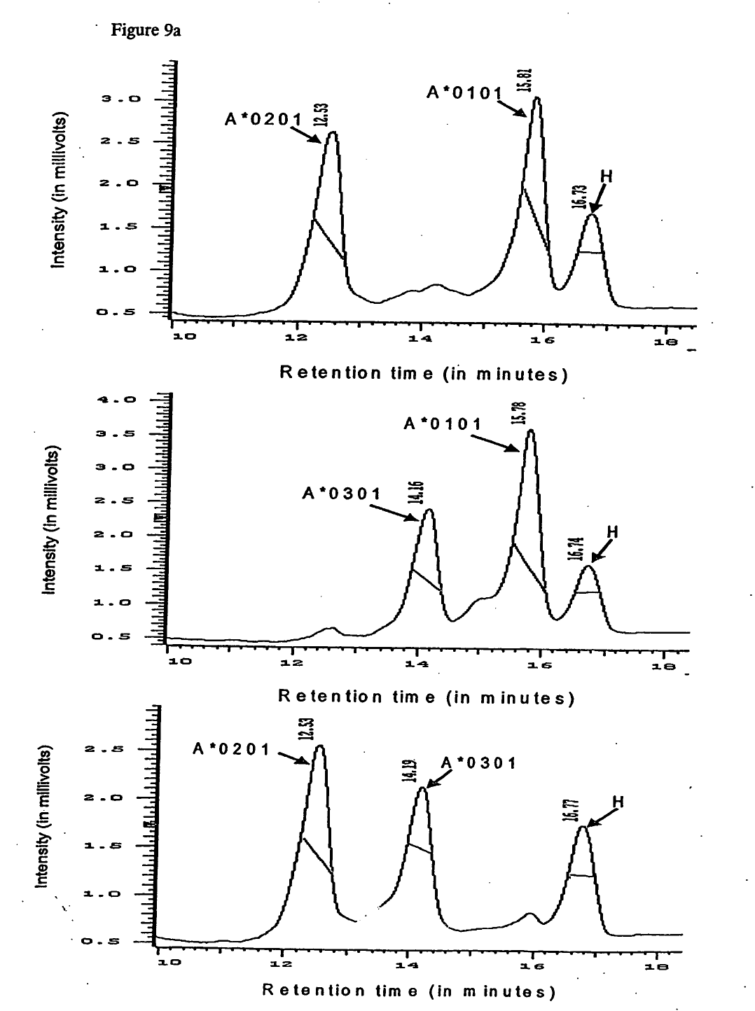

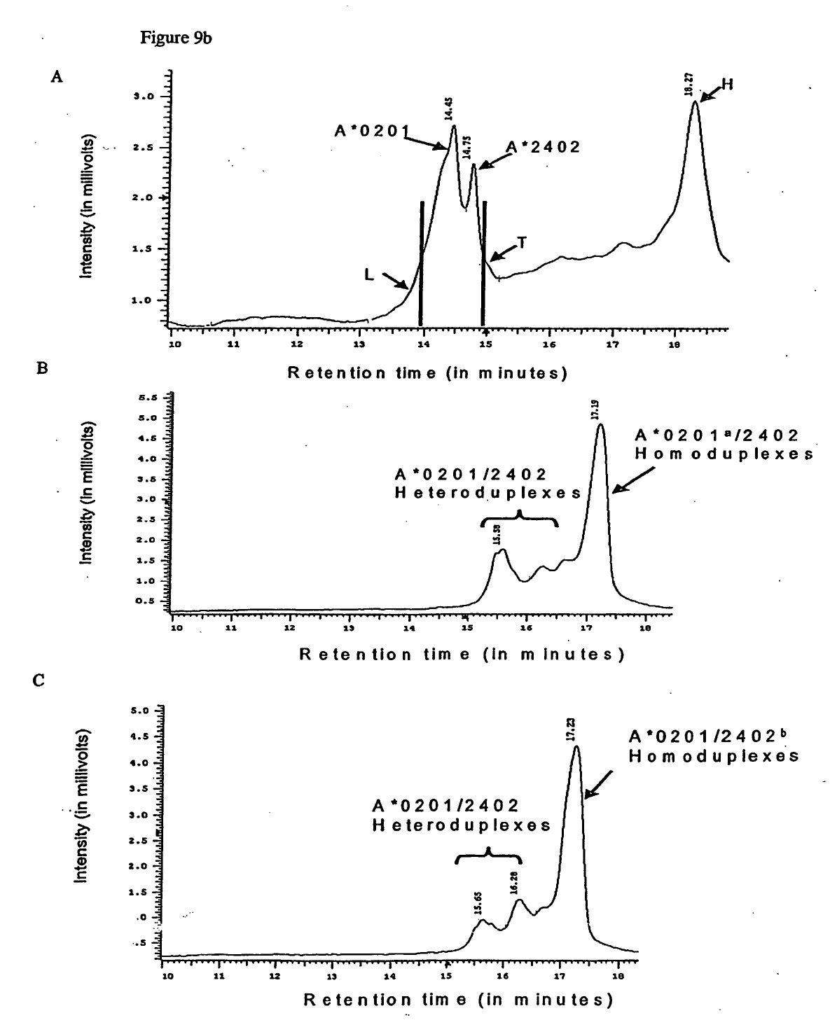

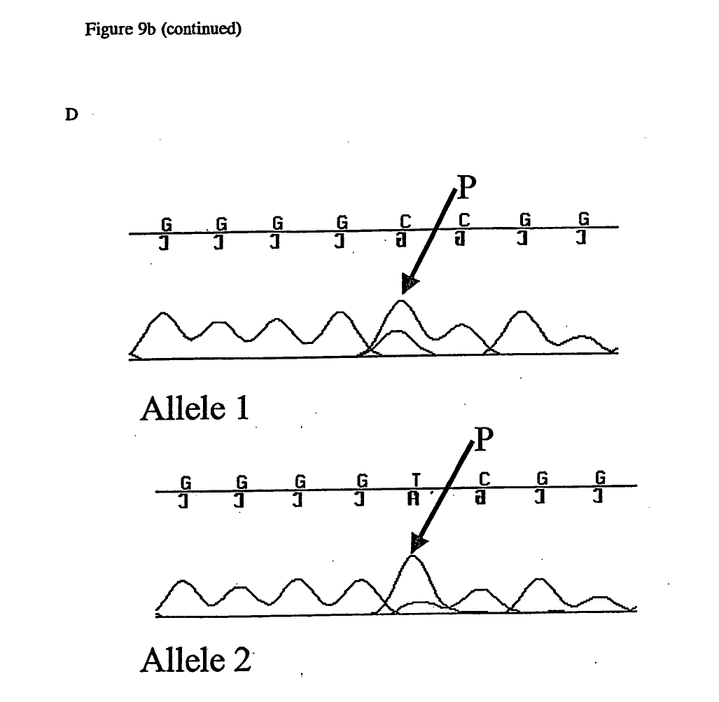

FIG. 9. Three well separated (FIG. 9a) and a closely separated (FIG. 9b) HLA-A heteroduplexes induced with TOPO-A*0101-mut13 reference probe from HLA-A heterozygotes. Black bars in panel A of FIG. 9b represent the region of the L (leading) and T (lagging) fractions that were collected for reamplification, while H indicates leftover homoduplexes. Panels B and C of FIG. 9b show the DHPLC resolution of reamplified A*0201- and A*2402 alleles respectively, from collected heteroduplex fractions and the effect of cross contamination of the heteroduplex fractions a and b are the dominating allele after reamplification. In panel D of FIG. 9b, effect of the cross contamination on sequence electropherograms is shown. P indicates a typical polymorphic site in the two alleles.

### EXAMPLES

Materials and Methods

DNA Samples

Genomic DNA was extracted according to standard methods (Maniatis T. et al., Molecular cloning: A laboratory manual. Cold spring Harbour, N.Y.: Cold Spring Harbour Laboratory, 1989) from International Histocompatibility Workshop (IHWS) B-lymphoblastoid cell lines (B-LCLs). DNA from patient samples was prepared according to the rapid lysis procedure of whole blood described by Huguchi et al (In:Erlich HA, ed. PCR Technology. New York. Stockton 1989:31-38).

Rapid Lysis Method of DNA Extraction

Extraction of genomic DNA is time consuming. Thus, for routine purposes, a rapid method for DNA preparation is useful. The DNA is released from the nuclei and made available for in vitro amplification, without removal of proteins and lipids. The PCR conditions given below are optimised for such rather crude DNA preparation.

Briefly, equal volumes of EDTA blood and ice cold solution A (320 mM sucrose, 10 mM Tris-HCl (pH7.5), 5 mM MgCl₂, and 1% Triton X-100) are mixed in a microcentrifuge tube and placed on ice for 10 minutes, followed by precipitation of nuclei at 13,000 rpm for 20 seconds. The pellet is washed and redissolved in solution B (10 mM Tris-HCl, 50 mM KCl, 0.1% Triton X-100 and 0.12 μg/μl proteinase K). The volume of the solution is adjusted with the same buffer to the initial volume of blood sample and incubated for 1 hour at 60° C. Proteinase K is thereafter inactivated by incubating the DNA solution at 95° C. for 10 minutes.

Sample PCR Conditions

PCR Mix:

(Per 100 μl total volume of PCR mixture which is sufficient for three samples)

| Final concentration | Volume of stock solution |
|---|---|
| dH₂O = | 69 μl |
| 1× NH4-buffer = | 10 μl 10× NH4-buffer |
| 0.2 mM dNTP = | 1.0 μl 20 mM dNTP |
| 0.5 μM HLA-AFN = | 2.5 μl 20 pmol/μl HLA-AFN |
| 0.5 μM HLA-ARN = | 2.5 μl 20 pmol/μl HLA-ARN |
| 0.25 μM HLA-AF = | 1.3 μl 20 pmol/μl HLA-ARN |
| 0.25 μM HLA-AR = | 1.3 μl 20 pmol/μl HLA-AR |
| 1 mM MgCl₂ = | 2.0 μl 50 mM MgCl₂ |
| 2 U/100 μl Tag (BioLine) = | 0.4 μl 5 U/μl Tag (BioLine) |
| Total Volume = | 90 μl |

10× NH₄ buffer: 160 mM (NH4)₂SO₄, 670 mM Tris-HCl (pH 8.8 at 25° C.), 0.1% Tween-20
Divide into three aliquots of 30 μl each.
Add 3 μl of crude DNA solution (as described above) or 30 ng of genomic DNA solution to each aliquot.
Subject the PCR mixture to the following cycling scheme in a PTC-200 PCR machine (MJ Research, USA: 95° C. 20 s, 65° C. 30 s, 72° C. 90 s for 35 cycles, then 72° C. for 8 min, before the reaction is ended at 4° C.).
Test the reaction mixture for PCR yields and specificity by agarose gel electrophoresis. A successful result should be a PCR band of approximately 980 bp and concentration ≧50 ng/μl (≧75 fmol/μl).

The primers used for PCR and mutagenesis of the reference probe are given in Table 1.

In all following procedures, it will be an advantage or a prerequisite that the PCR product is purified from primers and nucleotides. This is done according to the following protocol.

Purification of PCR Products:

Vortex Micro spin S-400 HR column (Amersham Pharmacia, Buckinghamshire, UK) vigorously until a homogenous suspension is obtained.
Loosen the screw cork by a quarter and break off the bottom seal of the spin column.
Place the column in an Eppendorf tube positioned in a micro-centrifuge. Spin for 1 minute at 3000 rpm to remove the storage buffer.
Discard the buffer and transfer the S-400HR column into a new and appropriately labelled Eppendorf tube.
Apply the sample PCR product (30 μl) carefully onto the top of the column bed. Spin for 2 minutes at 3000 rpm. Proceed with the primer-free PCR product, collected in the Eppendorf tube (approximately 35 μl).
   NB: Recommended volume of PCR product for best resolution by S-400HR columns is 25 μl-50 μl.
   Preparation of Single Stranded Reference Allele

In many of the allele separation methods disclosed herein it is desirable to obtain the reference DNA in a single stranded (ss) form. Two principal methods may be adopted for the preparation of Reference Probe ssDNA. One is based on Dynal streptavidin coated magnetic beads (M280). The other uses λ exonuclease to generate ssDNA. In both cases, WAVE separation is used to remove residual duplex DNA. Details of the procedures involved are described below:

Isolation of ssREF DNA using Dynal Streptavidin Coated Magnetic Beads (M280)

This technique involves amplification of the reference Probe with biotinylated HLA-AR-REF primer and unbiotinylated HLA-AF primer using the PCR scheme described in Example 1. Under appropriate conditions, the biotinylated Reference Probe is bound to the streptavidin of the Dynal magnetic beads (M280). The reference Probe is then denatured, leaving the sense strand bound to the magnetic bead while the antisense strand is released into the denaturation solution. With the help of a magnet stand, the solution containing the antisense of the Reference Probe is separated from the sense strand that is bound to the magnetic beads and collected for immediate usage. Additional purification steps, such as precipitation to remove NaOH, or restriction enzyme digestion with the appropriate enzyme, to remove residual double stranded reference probe could be applied. Furthermore methods to separate double stranded from single stranded DNA, such as ultracentrifugation or DHPLC separation could be used to further enrich for the antisense single stranded Reference probe.

Procedure

Gently resuspend 500 μl Dynal streptavidin coated magnetic beads (M280, 10 mg/ml) in an Eppendorf tube by pipetting.
Place on a magnet stand for 2 minutes. Pipette and discard the supernatant.
Remove the Eppendorf tube containing the magnetic beads from the magnet stand. Add 1 ml binding buffer and resuspend. (2× binding buffer: 6MLiCl in 1×TE buffer.)
Place the suspension on the magnet stand for 2 minutes. Pipette and discard the supernatant.
Remove the Eppendorf tube from the magnet stand. Add 1 ml binding buffer and resuspend the magnetic beads.
Divide the magnetic beads suspension into 2 aliquots of 500 μl each.

Add 500 μl of S-400HR column (Amersham-Pharmacia, UK) purified Reference Probe PCR products to each aliquot of the magnetic beads suspension.
Incubate the mixture at 43° C. for 15-60 minutes, under rotation in a hybridisation oven.
Place the mixture in the magnet stand. Carefully remove and discard the clear solution.
Add 100 μl freshly prepared 0.1M NaOH to the beads-DNA complex. Resuspend the complex in the solution by pipetting gently.
Incubate the suspension for 10 minutes at room temperature. Mix every two minutes.
Place the suspension in the magnet stand and collect the solution containing unbiotinylated ssDNA from the beads.
The single stranded reference DNA is now ready for usage or could be further purified as outlined above.
   λ Exonuclease Treatment of Duplex REF DNA to Generate ssREF DNA

In this technique the Reference Probe is amplified with 5′-phosphorylated HLA-AR-REF and non-phosphorylated HLA-AF-REF primers using the PCR scheme described in Example 1. Treatment of the resulting Reference Probe amplicons with λ exonuclease (USB Corporation, Cleveland, Ohio, USA), under the appropriate conditions of buffer and temperature, will lead to the selective digestion of the phosphorylated sense strand of the Reference Probe which was generated during PCR amplification. As a result, only the antisense strands of the Reference Probe are left as the final product. The antisense strand of the Reference probe is now ready for immediate usage. Additional purification steps, such as restriction enzyme digestion with the appropriate enzyme to remove residual double stranded reference probe could be applied. Furthermore methods to separate double stranded from single stranded DNA, such as ultracentrifugation or DHPLC separation could be used to further enrich for the antisense single stranded Reference probe.

Procedure

Purify 50 μg of the Reference Probe PCR product (i.e. 1000 μl successful PCR product) with the help of S-400 HR columns (Amersham Pharmacia, UK) as described above.
Divide the Reference Probe PCR product into 2 aliquots of 500 μl each.
Add 60 μl λ exonuclease buffer to each aliquot and adjust the volume to 595 μl with distilled water. (10×λ exonuclease buffer: 0.67M glycine-KOH, pH 9.3, 25 mM MgCl₂)
Add 5 μl λ exonuclease enzyme (10 U/μl) to each aliquot.
Mix and incubate at 37° C. for 20 minute.
Stop the reaction by heating at 75° C. for 10 minutes.
Pool the aliquots together in one Eppendorf tube.
The single stranded reference DNA is now ready for usage or could be further purified as outlined above.
   NB Five units of lambda exonuclease enzyme are sufficient to digest up to 2 μg of DNA.
   ds Reference Probe usually has a concentration of 50-100 ng/μl.

The two above described protocols can also be used to obtain sample alleles in a single stranded form if desired.

Annealing of Duplexes

Hybridisation to form homoduplexes and heteroduplexes was performed in a PTC-200 PCR machine (MJ Research, USA). The buffer used for hybridisation is either 1×SSC (15 mM NaCl, 15 mM Na Citrate), or 1×R2 buffer (50 mM NaCl, 50 mM Tris-HCl, pH8.0, 10 mM MgCl₂). The incubation temperatures were 95° C. for 4 minutes, −0.2°/s to 65° C. and 65° C. for 30 minutes in one cycle. Alternatively, hybridisation of heteroduplexes was also performed using two different heating blocks and an ice bucket, using the following incubation temperatures: 95° C. 4 min, 0° C. for 5 min, 65° C. for 30 minutes.

Heteroduplex Enrichment by Restriction Endonuclease

In this method of heteroduplex induction, the antisense strand of the reference probe prepared as described in Example 1 is added directly to dsDNA PCR product of the sample. Heteroduplexes between the antisense of the reference and the sense strand of the sample are enriched by Hae II endonuclease digestion of excess sample homoduplexes and heteroduplexes formed between the sample alleles (in the case of heterozygote samples).

Mix:

| | |
|---|---|
| Sample PCR product | 15 μl (0.05 μg/μl) |
| (0.75-1.0 μg) | |
| single stranded REF DNA | 5 μl (0.37 μg/μl |
| R2 Buffer | 2.3 μl |

(R2 Buffer: 50 mM NaCl, 50 mM Tris-HCl, pH 8.0, 10 mM MgCl₂)
Incubate the mixture at ₉₅° C. for 5 minute and immediately transfer to ice.
Subject to the annealing conditions described above.
Add 05 μl Hae II restriction endonuclease (20 U/11).
Incubate at 55° C. for 30 minutes. Heteroduplexes are generated and ready for separation by PAGE or WAVE.
   NB:
i 30° C. storage is recommended for samples that are not immediately resolved PAGE or WAVE. Also, stored samples should be defrosted on ice for separation.
ii Do not heat denature the enzyme.
   Heteroduplex Enrichment by λ Exonuclease

This method is based on enzymatic removal of one strand of duplex DNA that is phosphorylated in the 5′-end leaving the opposite strand as ssDNA. For this purpose, the sample PCR product is generated using 5′-phosphorylated HLA-AR primer leading to amplicons in which the antisense strand are 5′-phosphorylated and the sense strand is isolated by λ exonuclease digestion.

Alternative 1

Mix:

15 μl (0.75-1.0 μg or 1.2-1.5 pmol) purified sample PCR product
2 μl 10× λ exonuclease buffer
2 μl dH₂O
0.5 μl λ exonuclease (10 U/μl)
   Incubate at 37° C. for 20 minutes.
   Add 2,5 μl ssREF DNA (0,75-1 μg) (prepared as described above) and mix thoroughly.
   Add 2.5 μl R2 buffer (GIBCO) and subject to the annealing procedure described above. Heteroduplexes are generated and ready for separation by PAGE or WAVE.
      Alternative 2

In the absence of ssREF DNA, duplex Reference DNA (5′-phosphorylated sense strand) and sample DNA (5′-phosphorylated antisense strand) can be mixed and subjected to λ exonuclease digestion. ssREF DNA (antisense strand) and ssSAMPLE DNA (sense strand) are thus simultaneously generated.

Mix:

15 μl (0.75-1.0 μg or 1.2-1.5 pmol) purified sample PCR products
15 μl (0.75-1.0 μg or 1.2-1.5 pmol) purified Reference PCR
2.3 μl 10×λ exonuclease buffer
0.5 μl λ exonuclease (10 U/μl)
   Incubate at 37° C. for 20 minutes.
   Add 2.5 μl R2 buffer (GIBCO) and subject to the annealing procedure described above. Heteroduplexes are generated and ready for separation by PAGE or WAVE.
      Heteroduplex Induction by λ Exonuclease and Restriction Endonuclease

As described above, use of λ exonuclease results in the enzymatic removal of one strand of duplex DNA that is phosphorylated in the 5′-end leaving the opposite strand as ssDNA. Use of λ exonuclease can be combined with restriction enzyme digestion to result in a further optimisation of enzymatic enrichment of heteroduplexes, since neither the exonuclease nor the restriction endonuclease digestions are 100% efficient.

In this method, as described above the antisense strand of the reference probe prepared as described in Example 1 is added directly to dsDNA PCR product of the sample. However, in order for the λ exonuclease treatment to work the sample PCR product must be 5′ phosphorylated on the antisense strand. Heteroduplexes between the antisense of the reference and the sense strand of the sample are enriched by Hae II endonuclease, while treatment with λ exonuclease reduces the amount of homoduplexes formed by digesting the remaining single stranded sample DNA.

Mix:

| | |
|---|---|
| 5′ phosphorylated sample | 15 μl (0.05 μg/μl) |
| PCR product (0.75-1.0 μg) | |
| single stranded REF DNA | 5 μl (0.37 μg/μl |
| R2 Buffer | 2.3 μl |

R2 buffer: 50 mM NaCl, 50 mM Tris-HCl, pH 8.0, 10 mM MgCl₂
   Incubate the mixture at 95° C. for 5 minute and immediately transfer to ice.
   Subject to the annealing conditions described above.
   Add 0.5 μl Hae II restriction endonuclease (20 U/μl) and 0.5 μl λ exonuclease (10 U/μl, USB Corporation, Cleveland, Ohio, US).
   Incubate at 37° C. for 30 minutes. Heteroduplexes are generated and ready for separation by PAGE or WAVE.
      Heteroduplex Separation

The methods described below allow physical separation of sample alleles. Heteroduplexes generated between the sample alleles and the reference probe migrate differently in the separation media and may thus be separated from one another and remaining homoduplexes in the sample.

Native PAGE Separation and Elution of Heteroduplexes from the Gel

Separation of HLA-A heteroduplexes by nondenaturing polyacrylamide gel electrophoresis is based on the conformation differences induced in the DNA fragments as a result of the nucleotide mismatch(es) present in the heteroduplexes. Thus, HLA-A heteroduplexes of similar size but having different mutations in terms of number and position of mismatch(es) tend to display different migration characteristics when resolved through an appropriate native polyacrylamide gel medium (e.g. a 5% polyacrylamide gel containing 1.25% glycerol) by electrophoresis. Induction of heteroduplexes between sample alleles and the reference probe allows for separation of these heteroduplexes from sample alleles remaining as homoduplexes and heteroduplexes. Heteroduplexes induced between a heterozygote sample allele and the reference probe are also separated from one another.
Put together the glass plate sandwich as described by the manufacturer (Bio-Rad Inc., USA).

Prepare a nondenaturing polyacrylamide gel by mixing the following:

| | |
|---|---|
| Long Ranger polyacrylamide solution = | 5 ml |
| 10× TBE buffer = | 4 ml |
| 2% PEG-4000 = | 20 ml |
| 100% glycerol = | 0.6 ml |
| dH₂O = | 10.4 ml |
| Ammonium persulfate = | 0.2 ml |
| Total = | 40 ml |

Filter and degas the mixture using NALGENE® Disposable Filter Unit (Nalge Company, Rochester, N.Y., USA) for 15 minutes and add 40 μl APS (ammoniumpersulfate) and 5 μl TEMED.
Pour the solution into the glass plates sandwich (20 cm length×1 mm thickness), introduce the comb and allow to polymerise for 30 minutes.
Place the glass plates sandwich with the polymerised gel in the PROTEAN® II xi Cell electrophoresis chamber (Bio-Rad, USA).
Overlay the gel with 1×TBE buffer, remove the well comb and wash the wells by pipetting the buffer into the wells repeatedly.
Sample loading: Add 10 μl of ficoll loading buffer to 20 μl of the heteroduplex solution induced as described above.
Run the gel at 100V for 1 hr. then 300V for approximately 5 hrs.
Running temperature: 4-5° C. (maintained by constantly running ice-cold tap water through the electrophoresis chamber).
Turn-off the voltage supplies.
Stop the water flow, disconnect the hoses and empty the cooling chamber.
Carefully disassemble the gel sandwich from the electrophoresis chamber.
Detach the short plate from the sandwich.
Place the gel in 100 ml TBE buffer+5 μl EtBr solution (10 μg/μl) for 10 minutes
Detect the resolved bands by exposing the gel to UV-light.

10×TBE comprises 108 g Tris-HCl, 55 g boric acid, 8.3 g EDTA dissolved in 1000 ml of distilled water.

6×Ficoll loading buffer: 15% (w/v) Ficoll (Type 400), 0.25% (w/v) bromophenol blue, 0.25% (w/v) xylene in distilled water.

Elution of Resolved Heteroduplex from Polyacrylamide Gel for PCR Re-Amplification

Polyacrylamide gel segments containing the resolved heteroduplexes were cut from the polyacrylamide gel and rinsed in 400 μl of dH₂O. The supernatant was aspirated and discarded by pipetting. DNA fragments were eluted by adding 100 μl of dH₂O to the gel segment, shortly vortexing (5×), spinning at 3000 rpm for 1 minute and transferring the supernatant (containing the eluted DNA fragments) into a sterile micro test tube for storage and subsequent usage.

Resolution of Heteroduplexes by DHPLC

Heteroduplexes may be resolved by denaturating high pressure liquid chromatography (DHPLC). In the presence of appropriate buffer, the DNA binds to the DHPLC column. While running increasing amount of hydrophobic buffer over the column, DNA will be eluted off. Heteroduplexed DNA will bind more loosely to the column and will therefore be eluted off earlier than homoduplexed DNA.

The DNA sample is injected into the column of the DHPLC (Wave) instrument and the hydrophobicity of the buffer composition is gradually increased according to the scheme below (Universal gradient 6.1).

| TIME | % BUFFER | % BUFFER | FLOW | TEMP |
|---|---|---|---|---|
| (min) | A | B | (ml/min) | (° C.) |
| 0.0 | 65 | 35 | 0.9 | 61 |
| 0.1 | 60 | 40 | | |
| 17.0 | 28 | 72 | | |
| 17.1 | 0.0 | 100 | | |
| 18.1 | 65 | 35 | | |
| 18.2 | 65 | 35 | | |
| 20.2 | 65 | 35 | | |

Buffer A consists of: 100 ml 2M TEAA, 500 μl Acetonitrile (as an anti-bacterial agent) Adjust the volume to 2 l with HPLC-grade distilled water. Buffer B consists of 100 ml 2M TEAA, 500 ml Acetonitrile (Hyper grade), Adjust volume to 2 l with HPLC-grade distilled water.

Always run a blank sample (preferably water) as your first sample. This prepares the machine for separation and also cleans the column from DNA and enzyme residues from previous runs.

Run A*0101-reference DNA heteroduplex standard to determine the maximum retention interval that heteroduplexes will remain in the column before elution. This combination of A*0101 allele and Reference probe gives the lowest number of mutation sites; since the reference probe is derived from A*0101. Thus it elutes most closely to homoduplexes. Normal elution time will be between 16 and 17 minutes; depending on the state of the column.

Experience has shown A*02-REF heteroduplexes to be the fastest eluting species: eluting at around 12 minute retention time.

Thus, as a default setting for fragment collection, fractions should be collected every 30 seconds over the entire separation range of alleles, which for HLA-A with the given reference DNA is from 12 to 17.5 minutes elution time. For closely separating alleles, the configuration may be expanded within the elution interval of the alleles to facilitate distinctive collection of the leading and lagging fractions of the two separating alleles.

PCR Re-Amplification and Sequencing of Separated Heteroduplexes

Eluted heteroduplex DNA fragments were re-amplified by PCR. During reamplification, the end-mutations that were incorporated into the reference probe prevent synthesis of the reference probe in both primer sites from the heteroduplex template using the sample primers. Only the sample allele is amplified yielding pure homozygote sample amplicons.

Reamplification PCR mix (sufficient for 10 samples):

| Final concentration | Volume of stock solution |
|---|---|
| 1× PCR buffer: | 10 μl 10× PCR buffer |
| 0.20 mM dNTP: | 1 μl 20 mM dNTP |
| 0.2 μM HLA-AF | 1 μl 20 pmol/μl HLA-AF |
| 0.2 μM HLA-AR | 1 μl 20 pmol/μl HLA-AR |
| 1.5 mM | 3 μl 50 mM MgCl₂ |
| 2 U/100 μl: | 0.4 μl 5 U/μl Taq |
| dH₂O: | 83.5 μl |
| Total | 90 μl |

Mix thoroughly and divide into 10 aliquots at 9 μl each.

Add 1 μl solution of DNA eluted from polyacrylamide gel as described above or heteroduplex fraction collected from WAVE resolution to each aliquot.

Subject the mixtures to the PCR temperature cycling scheme used also for amplification of the samples as above, and test yields by agarose electrophoresis as described above.

The resulting DNA fragments were sequenced using intron 2 specific primers, fluorescent labelled ddNTP, and thermosequenase (Applied Biosystems, UK) according to the protocol given by the manufacturers. Automatic sequencing was performed in ABI PRISM 337™ sequencer (Perkin Elmer, USA), the resulting sequences were analysed by the WU-BLAST at Bork's group in EMBL (Heidelberg) programme at the IMGT-HLA web page (http:/www.expasy.ch/www/tools. htm similarity).

### Example 1

### Preparation of Enzyme Degradable Modified Reference DNA for HLA Class I Typing

In order to prepare a universal reference allele for HLA Class I typing the HLA-A locus was selected for analysis. All known alleles of HLA-A were aligned and regions that were conserved in exons 2 and 3 and also introns 1, 2 and 3 were found. These conserved regions were analysed for the presence of restriction sites which were present in all known alleles and which would result in the digestion of all the alleles into fragments substantially smaller than the full length allele. On carrying out this analysis it appeared that the restriction sites for the enzyme HaeII would be appropriate. In this regard it was found that HaeII had four restriction sites in the HLA-A amplicons. These restriction sites are conserved in virtually all known alleles and are present at positions 257, 520, 674 and 918 of HLA-A amplicons (as defined as the position at which the HaeII enzyme cuts). Four additional conserved regions were found in HLA-A alleles that would serve as alternative restriction sites for HaeII. These conserved regions contained partial HaeII sites and were located in the vicinity of the existing HaeII sites thereby allowing the mutation of the original site and at the same time create a new site. These alternative sites are located positions 255, 521, 676 and 916 of HLA-A amplicons. Finally, to prepare the modified reference allele the four inherent HaeII sites were deleted and the four alternative sites introduced by in vitro mutagenesis. The mutagenesis was done after the HLA-A*0101 allele was first amplified from a homozygous typing cell line (International Histocompatibility Workshop cell line IHWS 9041) using the HLA-AF-ref and HLA-AR-ref primers (see Table 1) under the PCR conditions outlined below, and the PCR product cloned into the pCR 2.1 Topo cloning vector from Invitrogen (http://www.invitrogen.com/catalog_project/cat_topota.html). This wild type HLA-A*0101 allele is already mutated at the position of the sample primers since the reference primers rather than the sample primers were used to clone the DNA fragment by PCR.

One clone was selected, and mutagenesis using the Quick change™ kit (Strategene, CA, USA, http://www.stratagene.com/mutagenesis/quikchng.htm) was performed. Briefly the plasmid was amplified with a pair of primers complementary to the desired mutation using Pfu Turbo polymerase from Stratagene, the nonmutated plasmid used as template for the reaction was digested by the dam methylation specific restriction enzyme DpnI, and the digested PCR product was directly transformed into Ca²⁺ competent E. Coli cells (pmos-blue from Amersham).

Success of mutagenesis was checked by amplifying the insert from several clones using the HLA-AF-ref and HLA-AR-ref primers, mixing the resulting PCR products with PCR products generated from the wild type clone using the same primers, and subjecting the mixture to digestion with HaeII. PCR products not being mixed with each other should yield the same restriction fragments as visualised by agarose gel electrophoresis, whereas in the case of successful mutation, the mixed PCR products should yield a longer fragment indicating that the heteroduplexes generated in the mixture were protected from digestion.

The four mutagenised HaeII sites were introduced successively into the mutated reference probe. The sequence of the mutated A*0101 reference allele (SEQ ID is NO. 1) is given in FIG. 1a. This reference allele is 985 bp in length.

Once prepared the modified reference allele could be used to form heteroduplexes—see Examples below.

General Conditions for Reference Probe PCR Amplification

Mix the following:

| | |
|---|---|
| dH₂O = | 836 μl |
| 10× PCR buffer = | 100 μl |
| 20 mM dNTP mix = | 10 μl |
| 20 pmol/μl AF-REF = | 10 μl |
| 20 pmol/μl AR-REF = | 10 μl |
| 50 mM MgCl_{2 =} | 30 μl |
| 5 U/μl Taq = | 4 μl |
| Total Volume = | 1000 μl |

Remove 50 μl for negative control.
Add 50 μl Reference DNA plasmid (1 ng/μl).
Distribute into 50 μl aliquots and subject to the following PCR scheme in a PTC-200 PCR machine (95° C. 20 s, 65° C. 30 s, 72° C. 90 s for 35 cycles, then 72° C. for 8 min, before the reaction is ended at 4° C.).
Pool the aliquots together.
Purify the PCR products and proceed to the next step.

10×PCR Buffer (detergent free): 100 mM Tris-HCl (pH 9.5 at 25° C.), 500 mM KCl.

### Example 2

### Increasing Numbers of Mutations in the HLA-A Reference Probe Result in Increased Separation Distance Between Homoduplexes and Heteroduplexes

Heteroduplexes were generated as described above using single stranded sample and reference DNA. Ref-250 is the HLA-A*0101 allele amplified with HLA-AF-ref and HLA-AR-ref primers and with the HaeII mutations depicted in FIG. 1b introduced at position 250. Similarly Ref-250/500 is the same as Ref-250 with HaeII mutations also introduced at position 500 etc. The reference probes Ref-250, Ref-250/500, Ref-250/500/910 and Ref-250/500/650/910 were used to generate heteroduplexes with sample DNA from a homozygous HLA-A1 sample and a heterozygous HLA-A1/A3 sample. These heteroduplexes were then subjected to separation using DHPLC as described above and the results are shown in FIG. 2b. It can be seen that increasing numbers of mutations in the HLA-A reference probe results in increased separation distance between homoduplexes and heteroduplexes. These results are shown graphically in FIG. 2c.

### Example 3

### Separation of HLA-A Alleles using DHPLC and Mutated Reference Probe

Double stranded and single stranded sample DNA was prepared from three heterozygous samples A*0101/A*0201 (A1/A2), A*0201/A*0301 (A2/A3) and A*0101/A*0301 (A1/A3) using appropriate PCR amplification conditions and primers as described above. Where single stranded (ss) DNA was used, this was obtained using PCR products where either the sense or the antisense strand was biotinylated. Removal of the biotinylated strand was performed using streptavidin M280 Dynabeads as described above. Single stranded reference DNA prepared as described above and in Example 1 was added to some samples—see below. Heteroduplexes were induced as described above and the samples were injected one by one into the column of the DHPLC apparatus. The results are shown in FIG. 3 where the intensity of the peaks eluted from the column as measured by UV light are shown. In panel a of FIG. 3, it can be seen that heteroduplexes in HLA-A heterozygous samples are not always observed. Double stranded (ds) PCR products of three different heterozygous samples are shown. No reference probe is added and it can be seen that in the A*010/A*0201 heterozygous (A1/A2) and the A*0201/A*0301 heterozygous (A2/A3) samples no heteroduplexes may be discerned. In the A*0101/A*0301 heterozygous sample (A1/A3) however, a heteroduplex peak may be seen. In panel b of FIG. 3, single stranded sense sample DNA is mixed with single stranded antisense reference DNA. Unlike the situation seen in panel a, heteroduplexes are observed in all the samples, and migrate and are separated in a reproducible manner according to the alleles present in the sample. Homoduplexes are also seen, indicating that the purification of the ss DNA, using biotinylated PCR products and magnetic beads, was not complete.

### Example 4

### Enrichment of Heteroduplexes by HaeII Digestion as Visualised with DHPLC Separation

FIG. 4 shows the results of an experiment where the formation of heteroduplexes is enriched by HaeII digestion. The experiment is carried out as described in Example 3 and the results visualised with DHPLC separation using the Wave equipment. In some samples however the restriction enzyme HaeII is added as described in the Materials and Methods section above.

Panel a of FIG. 4 shows the enrichment of heteroduplexes of A1*0101 by HaeII digestion. In this panel it can also be clearly seen that the reference probe is not purely single stranded, since the heteroduplex generated between ass A1 and sss ref DNA (marked with the left stipled line) is visible also in the panel with digested ds A1 with ass ref DNA where the heteroduplex between sss A1 and ass ref DNA is dominating (marked with the right stipled line). In panel b the same three heterozygous combinations from FIG. 3 are shown after heteroduplex induction and HaeII digestion for enrichment of heteroduplexes. The major peaks are digested DNA, the heteroduplex peaks are rather weak, indicating that the induction of heteroduplexes in this experiment was not optimal. Panel c is the same as panel b in FIG. 3, and is included to allow direct comparison of peak migration.

### Example 5

### Induction of HLA-A Heteroduplexes by Modified HLA-A Reference Allele which Inhibits the Digestion of Heteroduplexes by HaeII Endonuclease

Sample DNA of HLA-A*0101 exon2 and exon3 was prepared using the HLA-AF and HLA-AR primers (see Table 1 and Cereb et al., Tissue Antigen, 1995, 45:1-11) yielding a PCR product of 985 bp. A similar 985 bp fragment of reference DNA was prepared by cloning HLA*0101 after amplification with HLA-AF-ref and HLA-AR-ref primers followed by in vitro mutagenesis of all four conserved HaeII sites present within the amplified fragment by moving the HaeII site a few base pairs.

To enrich for heteroduplexes representing only one of the strands in the sample DNA, single stranded reference DNA was prepared and added to double stranded sample DNA in increasing amount. Heteroduplexes were allowed to form in 50 mM Tris pH 8.0, 10 mM MgCl2 and 50 mM NaCl by heating the sample to 95° for 5 min. The samples were then put on ice, followed by hybridization at 65° C. for 30 mins. The sample was then put on ice, 10 U of HaeII was then added to each of the samples, and samples were further incubated at 37° C. for 1 hour or at 55° C. for 30 mins.

DNA was visualised with ethidium bromide staining after agarose electrophoresis. Complete digestion of the sample DNA was observed when single stranded reference DNA was not added (lane 10, FIG. 5), whereas increasing amount of full length heteroduplexes are observed with the addition of increasing amounts of single stranded reference DNA (lanes 6 to 9, FIG. 5). (The numbers above the lanes refer to the relative amount of single stranded reference DNA in relation to the fixed amount of double stranded sample DNA used in all 10 lanes). Both the reference DNA and sample DNA but not heteroduplexes formed between reference DNA and sample DNA will be digested by HaeII into fragments of approximately 180-250 bp in length.

It is important to note that because an agarose gel is used in this experiment separation of the heteroduplexes and homoduplexes is not observed in lanes 1 to 5. This is because an agarose gel does not normally separate between heteroduplexes and homoduplexes, unless the heteroduplexes are really open, or the agarose gel of really high quality. FIGS. 4a and 4b however show the results from a DHPLC Wave experiment with HaeII digestion of duplexes. In these experiments the heteroduplexes and homoduplexes are separated.

Additional Materials and Methods Used in Example 6

DNA Samples

Genomic DNA was extracted according to standard methods (Maniatis et. al., Molecular Cloning: A laboratory manual, Cold Spring Harbour, N.Y., 1989) from International Histocompatibility Workshop B-lymphoblastoid cell lines (IHWS-B-LCLs). A total of 90 DNA samples from healthy individuals prepared according to the rapid lysis procedure of whole blood described by Higuchi (Erlich HA, ed. PCR technology, NY, Stockton, 1989) was kindly provided by the Norwegian Bonemarrow donor register (NBMR). Also, 11 additional DNA materials for the external proficiency testing on typing and crossmatching (Eurotransplant Reference Laboratory for Histocompatibiliy, Leiden, The Netherlands) hereafter referred to as EPTTC-DNA were analysed. Among the total 101 samples analysed, 15 were carefully selected on the basis of their HLA-A heterozygous combinations.

In Vitro Amplification of HLA-A Alleles

A 985 bp fragment containing HLA-A exon 2 and exon 3 was amplified from genomic DNA, plasmid DNA or eluates of heteroduplexes (see below) using specific HLA-A primers modified as indicated in Table 1. PCR conditions were as follows: 0.2 μM each of the specific primers, 0.2 mM deoxyribonucleotide triphosphate (dNTPs), 1× NH₄-buffer (16 mM (NH4)₂SO₄, 67 mM Tris-HCl (pH 8.8 at 25° C.), 0.1% Tween-20) (BioLine, UK), 1.0 MgCl₂ and 2 U/100 μl of Taq polymerase (BioLine, UK). For amplification of the samples, 50 ng genomic DNA was amplified in a 50 μl volume using appropriately end-labelled HLA-AF and non-labelled HLA-AR primers together with 0.4 μM HLA-AFN and HLA-ARN (table 1). For amplification of the reference probe, 10 ng of plasmid DNA was amplified in a 50 μl volume using non-labelled HLA-AF-ref and appropriately labelled HLA-AR-ref primers. For reamplification of heteroduplex eluates, 5 μl eluate was amplified in a 50 μl volume using HLA-AF and HLA-AR primers. Temperature cycling was as follows: 95° C. for 20 seconds, 65° C. for 30 seconds, and 72° C. for 90 seconds for 35 cycles and final extension at 72° C. for 8 minutes. UV light detected the PCR yields after ethidium bromide staining of 1.5% agarose gel containing amplicons resolved by electrophoresis. Amplicons were purified using Microcon®-PCR Filter Units (Millipore Corporation, Bedford, Mass., USA), re-resolved by 1.5% agarose gel electrophoresis before ethidium bromide staining and quantitation that was facilitated by UV-light detection of resolved amplicons. Quantitation of amplicons was performed by the Quantity One® Quantitation software (Bio-Rad Laboratories Inc., CA, USA). The φ 74 DNA/BsuRI (Hae II) Marker, 9 DNA quantitation and molecular weight marker (MBI Fermentas Inc., Burlington, ON, Canada) that was resolved along with the amplicons served as standard.

Preparation of Reference Probe

The reference probe was prepared in a similar way to that described in Example 1. Specifically, a 985 bp fragment of HLA-A*0101 exon 2 and 3 was amplified by PCR from genomic DNA of the IHWS-BCL-9043 using the HLA-AF-ref and HLA-A AR-ref primer (table 1). The resulting amplicons were cloned into PCR®4-TOPO® plasmid (Invitrogen Corporation, Carlsbad, Calif., USA). Mutations as described in Example 1 were introduced into the cloned HLA-A*0101 fragments at non-polymorphic positions using the Quickchange™ Site-Directed Mutagenesis kit (Stratagene, CA, USA). A total of thirteen substitutions at four conserved sites were introduced successfully into the finished mutated HLA-A*0101 reference probe. The resulting recombinant plasmids were designated TOPO-A*0101-mut2-13 respectively and the sequences verified by sequencing. The sequence of the mutated HLA-A*0101 reference probe is shown in SEQ ID No. 1.

Generation of Heteroduplexes

Single stranded DNA (ssDNA) was obtained either by magnetic beads separation essentially as described by Arguello et. al., Nucleic Acids Research, 25, pg 2236-2238 (1997), or by λ-exonuclease digestion. For magnetic beads separation, equal amounts (200 ng) of in vitro amplified 5′-biotinylated reference double stranded DNA (dsDNA) and sample dsDNA were mixed in 80 μl volume. M280 streptavidin dynabeads (40 μl) (Dynal, Oslo, Norway) was washed and resuspended in 80 μl freshly prepared 6 mM LiCi/TE solution. The PCR mixture was added to the Dynabeads suspension and incubated at 43° C. for 30 minute under rotation in a hybridisation oven/shaker (Amersham plc, UK). Non-biotinylated DNA strands were isolated by denaturation in 0.1M NaOH, removal of the beads and precipitation in three volumes of ethanol by spinning for 30 minutes at 10,000 g and 4° C. The precipitate was washed in 70% ethanol and dissolved in 20 μl 1×R2 buffer (5 mM NaCl, 5 mM Tris-HCl, pH 8.0, 1 mM MgCl₂) (Life Technologies Inc., Rockville, USA). For λ-exonuclease digestion, equimolar amount (200 ng each) of in vitro amplified 5′-phosphorylated reference amplicon and sample amplicon were mixed. The samples were digested in 1×λ-exonuclease buffer (67 mM glycine-KOH (pH 9.3), 2.5 mM MgCl₂) and 10 Units of λ-exonuclease (USB Corporation, Cleveland Ohio, USA) in 20 μl total reaction volume for 15 minutes at 37° C. Thereafter, 2 μl 10×R2 (50 mM NaCl, 50 mM Tris-HCl, pH 8.0, 10 mM MgCl₂) (Life Technologies Inc., Rockyille, USA) buffer was added before heteroduplex induction. Induction of heteroduplexes was performed in a PTC-200 PCR machine (MJ Research, USA) as follows: Incubation at 95° C. for 2 minutes followed by incubation at 65° C. for 15 minutes and rapid cooling to 4° C.

Allele Separation by DHPLC

Heteroduplexes were separated using DHPLC by resolving 20 μl of the heteroduplex solutions by the WAVE™ DNA Fragment Analysis System (Transgenomic, Inc., San Jose, Calif., USA). Separation was performed at 61° C. using the universal gradient for mutation detection on the WAVE. Separated heteroduplexes were automatically collected in 200 μl volumes by the fragment collector accessory of the DHPLC machine WAVE™ DNA Fragment Analysis System (Transgenomic, Inc., San Jose, Calif., USA). For reamplification of separated alleles, 5 μl of the eluate was used as template in 50 μl PCR reaction volume as described.

Sequencing of Reamplified Amplicons

HLA-A allele amplicons obtained from the reamplification of resolved heteroduplexes were sequenced with HLA class I-intron II specific primers, HLA-AF-SEQ, and HLA AR-SEQ primers respectively using the CEQ 2000 chemistry on an automatic sequencer (CEQ 2000 sequencer, Beckman Coulter Inc., CA, USA). The resulting sequences were analysed by the Sequencher™ software (Genes Codes Corporation, Ann Arbor, Mich., USA) and HLA-A alleles determined by the IMGT/HLA DNAPLOT SEARCH programme at the IMGT-HLA web page (http://www.ebi.ac.uk/imgt/hla/dnaplot.html).

### Example 6

### Sequencing Based Typing of Human Leukocyte Antigens Class IA (HLA-A) Alleles Separated by Denaturing High Performance Liquid Chromatography

This example demonstrates that HLA-A alleles may be physically separated using a mutated reference probe and DHPLC prior to sequencing. The method will allow for development of fully automated sequencing-based genotyping.

λ-Exonuclease Generation of Single Stranded DNA

In order to limit the number of heteroduplex DNA species formed between the reference probe and sample alleles, we investigated whether single stranded (ssDNA) suitable for heteroduplex induction could be generated by λ-exonuclease digestion of the 5′-phosphorylated sense strand of the sample's double stranded DNA (dsDNA) and the antisense strand of the dsDNA reference probe (S1 (step 1) of FIG. 6). We found that λ-exonuclease was very efficient in generating ssDNA suitable for heteroduplex induction.

A HLA-A Reference Probe Refractory to Amplification

Different methods that are based on heteroduplex conformation have been described and applied for separation of polymorphic alleles (Arguello et. al., Proc. Natl. Acad. Sci., USA, 93, pg 10961-65, 1996, Supra, 1997, Tissue Antigens, 52, pg 57-66, 1998, Eberle et. al. Tissue Antigens, 49, pg 365-375 1997, Wang et. al. Tissue Antigens, 2, pg 134-140 1997, Knapp et. al. Tissue Antigens, 50, pg 170-177 1997, FIG. 6).

However, to obtain alleles that are separated not only from each other, but also from the reference probe, a reference DNA that is refractory to reamplification is required. For heteroduplex based separation of HLA-A alleles using HLA-A*0101 as reference allele (Arguello et. al, Supra 1997), we therefore designed primers identical to the HLA-A specific primers (Cereb et. al. Tissue Antigens, 45, pg 1-11 1995), but extended them 5-6 bases in the 3′ end (table 1). The primers contained a mismatched base corresponding to the 3′ end of the HLA-A specific primers. We found that these primers could be used to amplify HLA-A alleles directly from genomic DNA. Furthermore, the reference probe generated with these reference primers could not be amplified using the HLA-A specific primers (S2 (step 2) of FIG. 6). By applying a reference probe with this characteristic for the separation of HLA-A alleles, we were able to selectively amplify only the sample alleles in separated heteroduplexes (Arguello et. al., Supra, 1997, S3 (step 3) of FIG. 6). To avoid interference from the reference DNA during sequencing, Arguello et. al. 1997 introduced mutations into the primer site of the reference allele by performing a second nested PCR. In this study we introduced mutations into both ends of the reference DNA already at the initial amplification.

A Reference Probe for HLA-A Separation by DHPLC

Heteroduplexes may be separated by various media such as polyacrylamide gels or by DHPLC. To develop an efficient method for separation of HLA-A alleles and subsequent genotyping by sequencing, we applied the WAVE system DHPLC. DHPLC allows for the detection of heteroduplexes at a specific temperature, depending on the size and nucleotide composition of the DNA fragment (Underhill et. al. Genome Research, 7, pg 996-1005 1997 and Liu et. al. Nucleic Acids Research, 26, pg 1396-1400 1998). When using A*01 01 allele as a reference, we were able to resolve a single heteroduplex peak from homoduplexes at 61° C. using the universal gradient for mutation detection by the WAVE. To facilitate the separation of HLA-A heteroduplexes from one another and from homoduplexes in a given sample by DHPLC, we introduced mutations into the conserved domains of the HLA-A*0101 fragment as described in the Materials and Methods section above. We postulated that these mutations in combination with subsequent inherent mutations in heteroduplexes generated between the reference probe and sample alleles (S2 (step 2) of FIG. 6) would facilitate separation of heteroduplexes from one another during DHPLC resolution. A total of 13 mutations located at four conserved regions in HLA-A alleles were introduced into the 985 bp fragment by successive in vitro mutagenesis. The final resulting fragment was designated TOPO-A*0101-mut13 reference probe.

The efficiency of the reference probe for separation of HLA-A alleles by DHPLC was tested by inducing heteroduplexes between the four different reference probe constructs representing 1, 2, 3 and 4 mutated regions and HLA-A amplicons from International Histocompatibility Workshop B-lymphoblastoid cell lines (IHWS-B-LCLs) 9043 (A*0101), 9007 (A*0201) and 9005 (A*0301) and their combinations as artificial heterozygotes (FIG. 7). The HLA-A heteroduplexes induced by the various reference probe constructs eluted from the column in a pattern that reflected the influence of the number and positions of mutations that were incorporated into the respective reference probes. Importantly, heteroduplexes formed between the respective reference probe constructs and sample 9043 (HLA-A*0101) amplicons could be separated from the HLA-A*101 homoduplexes (FIG. 7).

An additional advantage with incorporation of mutations into the reference DNA is that these mutations will induce mismatches in the heteroduplexes of all known alleles at the given locus. The possibility that a heterozygous sample will be misinterpreted as a homozygous in the case of shared identity between one allele and the reference (Arguello et. al., Supra, 1998) is therefore eliminated.

DHPLC-SBT of HLA-A Alleles Using the TOPO-A*0101-mut13 Reference Probe

To assess the robustness of the DHPLC-SBT method, we applied it for sequencing of 101 DNA samples. Among these, 86 samples were randomly selected, while 15 heterozygous samples were carefully selected based on the data from our pilot experiments which showed that these HLA-A heterozygous combination tend to separate closely during DHPLC resolution (separation interval less than 0.5 minutes). In total, 80 samples were typed to be heterozygotes, while 21 samples were typed as homozygotes. A total of 20 HLA-A alleles representing 9 HLA-A generic groups (panel a of FIG. 8) and 34 heterozygous combinations of HLA-A alleles (panel b of FIG. 8) were successfully typed by DHPLC-SBT.

During DHPLC resolution, heteroduplexes that were generated between the sample alleles and TOPO-A*0101-mut13 reference probe from 60 of the 80 heterozygous samples were well separated (FIG. 9a) and easily collected for reamplification and subsequent sequencing. Upon sequencing of the reamplified alleles, the presence of only one allele in each heteroduplex peak could be verified and the allele determined. In the remaining 20 heterozygous samples, heterozygosity of the sample could be observed, but the two peaks were eluted so close to each other that they were partly overlapping (separation interval <0.5 minute, panel A of FIG. 9b). By collecting the leading part (L) of the first eluting peak and the trailing part (T) of the second (panel A of FIG. 9b), we obtained samples that were sufficiently enriched to be typed by sequencing. Although cross contamination of eluting DNA species in collected eluates of the closely separated heteroduplexes occurred (panel B and C of FIG. 9b), the allele could easily be identified as the dominating peak in the sequence electropherogram (panel D of FIG. 9b). To ensure that only the leading or the lagging fraction of the respective eluted heteroduplexes were collected, we optimised the fragment collection parameters of the fragment collection accessory of the WAVE™ machine. Using this approach, we could successfully collect pure heteroduplex eluates from the closely separated heteroduplex species with the corresponding sequence electropherograms showing no sign of cross contamination (data not shown).

On comparing the sequencing data of heterozygous samples, it appeared that the separation interval between heteroduplex peaks of each sample correlated with the number of mismatched nucleotide positions between the two separating heteroduplex species (panel b of FIG. 8). The higher the difference in the number of mismatches between two heteroduplex species of a heterozygote sample induced with TOPO-A*0101-mut13 reference, the larger the elution interval that was recorded in DHPLC separation.

In general, the HLA-A sequence as defined by the WAVE MAKER software was highly GC-rich (68%). It has regions with varied melting temperature within the fragment. Moreover, the analysed DNA fragment is 985 bp long. These characteristics does not favour detection of mutations (Underhill et. al., Supra, 1997) lest physical separation of alleles by DHPLC. We have shown that incorporation of evenly spaced mutations in the reference DNA at conserved positions facilitated the separation and collection of HLA-A gene fragments containing exon 2, intron 2 and exon 3 by DHPLC. We have also shown that separated HLA-A alleles are successfully typed by sequencing. In conclusion, incorporation of uniformly distributed mutations in the reference DNA facilitated DHPLC-SBT of HLA-A alleles. Application of this technique will greatly enhance the quality of genomic typing of polymorphic gene loci since alleles can be identified with certainty through sequencing without the prior need for allele specific amplification or cloning.

TABLE 1

| | | | | |
|---|---|---|---|---|
| | | | Nucleotide | |
| Oligo | Sequence | Location | postion^{a} | Purpose |

Published primers (Cereb et al)

| | | | | |
|---|---|---|---|---|
| HLA-AF | 5′-GAAACSGCCT CTGYGGGGAG AAGCAA | intron 1 | 310-335 | Sample PCR |
| HLA-AR | 5′-TGTTGGTCCC AATTGTCTCC CCTC | intron 3 | 1271-1294 | Sample PCR |
| HLA-clI-ex3 | 5′-ACCCGGTTTC ATTTTCRGTT | intron 2 | 1139-1158 | Sequencing PCR |
| HLA-cli-ex2 | 5′-AACYGAAAAT GAAACCGGGT | intron 2 | 1139-1158 | Sequencing PCR |

Primers designed buy us (unpublished):

| | | | | |
|---|---|---|---|---|
| HLA-AF- | 5′-phospo-GAAACSGCCT CTGYGGGGAG AAGCAA | intron 1 | 310-335 | Sample PCR |
| phospho | | | | |
| HLA-AF- | 5′-biotin-GAAACSGCCT CTGYGGGGAG AAGCAA | intron 1 | 310-335 | Sample PCR |
| biotin | | | | |
| HLA-AF-ref | 5′-GAAACNGCCT CTGYGGGGAG AAGCAgCGGG CC | intron 1 | 310-340 | Reference PCR |
| HLA-AF-ref | 5′-biotin-TGTTGGTCCC AATTGTCTCC CCTgCTTGTG | intron 3 | 1265-1294 | Reference PCR |
| biotin | | | | |
| HLA-AR-ref | 5′-phospho-TGTTGGTCCC AATTGTCTCC CCTgCTTGTG | intron 3 | 1265-1294 | Reference PCR |
| phospho | | | | |
| HLA-AFN | 5′-NNNNNNNNNN NNGGGGAGAA GCAddA | intron 1 | 312-335 | Sample PCR, |
| | | | | competitor primer |
| HLA-ARN | 5′-NNNNNNNNNN NNTTGTCTCC CCTddC | intron 3 | 1271-1294 | Sample PCR, |
| | | | | competitor primer |
| hlaa-p250F | 5′-G AAGATGGAG CCGGGCGCCC CGTGGATAGA GC | exon 2 | 547-578 | mutagenesis |
| hlaa-p250R | 5′-GCTCTATCCA CGGGGCGCCC GGCTCCATCT TC | exon 2 | 547-578 | mutagenesis |
| hlaa-p500F | 5′-AAGATGGAG CCGGGCGCCC CGTGGATAGA GC ATTTTC | intron 2 | 814-849 | mutagenesis |
| hlaa-p500R | 5′-GAAAATGAAA CCGGGTAAAG CGCCCTGGGC CTCTCCC | intron 2 | 814-849 | mutagenesis |
| hlaa-p650F | 5′-GGGCCGGAAG CGCTCTTCCT CCGCGG | exon 3 | 972-996 | mutagenesis |
| hlaa-p650R | 5′-CCGCGGAGGA AGAGCGCTTC CGGCCC | exon 3 | 972-996 | mutagenesis |
| hlaa-p910F | 5′-CAGGGGCCAC GGCGCTCCTC CCTGATCGC | intron 3 | 1211-1239 | mutagenesis |
| hlaa-p910R | 5′-GCGATCAGGG AGGAGCGCCG TGGCCCCTG | intron 3 | 1211-1239 | mutagenesis |

1. A method of separation of alleles in a sample, comprising the use of a modified reference allele, wherein said modified reference allele comprises one or more modifications such that heteroduplexes formed between said reference allele and a sample allele present in the sample are resistant to digestion with one or more endonuclease enzymes which digest homoduplexes or heteroduplexes of said sample alleles.

2. The method of claim 1, wherein said modified reference allele is single-stranded.

3. The method of claim 1, wherein either or both of said sample allele and reference allele is in double stranded form and is rendered single stranded by specific digestion of one of the strands of said allele.

4. The method of claim 3, wherein said strand which is digested is labeled to permit said digestion.

5. A method of separation of sample alleles in a sample, comprising the use of a reference allele and further comprising the use of either or both of a double stranded sample allele and said reference allele in a double stranded form, wherein one of the strands of the double stranded allele present is labeled so as to allow specific digestion of one of the strands.

6. The method of claim 5, wherein said reference allele is modified to comprise one or more modifications such that heteroduplexes formed between said reference allele and a sample allele present in the sample are resistant to digestion with one or more endonuclease enzymes which digest homoduplexes or heteroduplexes of said sample alleles.

7. The method of claim 1, wherein said endonuclease enzymes also digest homoduplexes of said reference alleles.

8. The method of claim 1, wherein said endonuclease enzymes are restriction enzymes.

9. The method of claim 3, wherein said specific digestion is achieved by use of an enzyme.

10. The method of claim 9, wherein said enzyme is λ-exonuclease and said strand which is specifically digested is 5′-phosphorylated.

11. The method of claim 3, wherein both the sample and reference alleles are provided in a double stranded form, and either the sense or the antisense strand of the sample alleles is labeled with a 5′-phosphorylated group and the opposing strand of the double stranded reference allele is labeled with a 5′-phosphorylated group.

12. The method of claim 3, wherein only one of the sample and reference alleles is supplied in a double stranded form and one of the strands is phosphorylated at the 5′ end.

13. The method of claim 3, wherein said reference allele is single-stranded and said sample allele is double-stranded.

14. The method of claim 1, wherein said reference allele comprises a modification which induces a mismatch between a sample and reference allele.

15. The method of claim 14, wherein at least 1% of the total number of bases in the reference allele are modified to form the modified reference allele.

16. The method of claim 1, wherein in said modified reference allele, a restriction site for at least one restriction enzyme has been removed or inactivated, and introduced at a different location.

17. The method of claim 1, wherein said duplex molecules and digested molecules are separated using separation means which physically separate molecules on the basis of one or more of size, conformation, hydrophobicity and charge.

18. The method of claim 17, wherein said separation means is selected from the group consisting of polyacrylamide gel electrophoresis (PAGE), denaturing high performance liquid chromatography (DHPLC), capillary electrophoresis and mass spectrometry.

19. The method of claim 1, wherein the sample strand of a heteroduplex formed between said sample and reference alleles is amplified following separation of said heteroduplex.

20. The method of claim 19, wherein said reference allele is further modified at a site corresponding to a primer binding site in a sample allele so as to prevent or disrupt binding of a sample amplification primer for said sample allele to said reference allele.

21. The method of claim 19 wherein a competitor primer is used to suppress amplification of the reference strand.

22. A method of genotyping the alleles present in a biological sample, comprising subjecting said sample to the method of allele separation of claim 1, separating the heteroduplexes formed between sample and reference alleles in a separation medium and identifying the alleles present, wherein the alleles present are identified by the migration pattern of the separated heteroduplexes in or on the separation medium, by direct sequencing of the sample alleles in the separated heteroduplexes, or by both the migration pattern of the separated heteroduplexes and direct sequencing of the sample alleles in the separated heteroduplexes.

23. The method of claim 22, wherein the method of genotyping the alleles present in a biological sample is used in HLA typing, determination of polymorphisms involved in metabolism of pharmaceuticals, determination of mutations in disease loci, determination of mutations in cancers, or determination of viral variants in chronic viral diseases.

24. A method of diagnosis of disease in a subject, or the susceptibility of a subject to a disease, comprising subjecting a nucleic acid sample of said subject to the method of allele separation of claim 1, and carrying out genomic typing to determine whether at least one particular mutation is present.

25. The method of claim 22, wherein the migration of the heteroduplexes in the separation medium is monitored or detected by ethidium bromide staining, detection of fluorescently-labeled alleles or by detecting elution position of column peaks.

26. A modified reference allele which has been modified to delete or inactivate at least one endonuclease site which is present in a corresponding sample allele, and to re-introduce said endonuclease site at a different location, which is not present in said sample allele.

27. A method for preparing a modified reference allele, said method comprising the steps of (i) selecting a sample allele, (ii) identifying one or more endonuclease enzymes which cleave all known sample alleles at least once, (iii) deleting or inactivating these endonuclease sites, and (iv) introducing one or more alternative sites for the same enzyme into the allele, thereby forming a modified reference allele.

28. The modified reference allele of claim 27, further comprising a modification at a site corresponding to a primer binding site in said sample allele so as to prevent or disrupt binding of a sample amplification primer for said sample allele to said reference allele.

29. The modified reference allele of claim 26, wherein said modified reference allele is an HLA allele.

30. The modified reference allele of claim 29, wherein said modified reference allele is a nucleic acid molecule comprising a nucleotide sequence of SEQ ID NO. 1, or a fragment thereof comprising a functionally active sequence, or a sequence which is degenerate, substantially homologous with, or which hybridizes with the sequence of SEQ ID NO. 1 or with the sequence complementary thereto, or a fragment thereof encoding a functionally active product.

31. A kit for use in allele separation or typing comprising the modified reference allele of claim 26, and one or more further components selected from the group consisting of sample primers to amplify the particular sample alleles concerned, reference primers to amplify the modified reference allele, a restriction enzyme, and an exonuclease.

32. A method of separation of alleles in a sample, wherein the alleles are separated by denaturing high-performance liquid chromatography (DHPLC), comprising the use of a modified reference allele which forms heteroduplexes with sample alleles contained within the sample and which contains a plurality of additional modifications such that improved separation of alleles is obtained.

33. The method of claim 32, wherein said reference allele comprises a modification which induces a mismatch between a sample and reference allele.

34. The method of claim 33, wherein at least 1% of the total number of bases in the reference allele are modified to form the modified reference allele.

35. The method of claim 32, wherein the difference in retention time on said DHPLC between different homo- and heteroduplexes is at least one minute.

36. The method of claim 32, wherein the elution profile of said DHPLC is used to genotype unknown samples independently of sequence analysis by comparison with known sample profiles.

37. The method of claim 6, wherein said endonuclease enzymes also digest homoduplexes of said reference alleles.

38. The method of claim 6, wherein said endonuclease enzymes are restriction enzymes.

39. The method of claim 5, wherein said specific digestion is achieved by use of an enzyme.

40. The method of claim 39, wherein said enzyme is λ-exonuclease and said strand which is specifically digested is 5′-phosphorylated.

41. The method of claim 5, wherein both the sample and reference alleles are provided in a double stranded form, and either the sense or the antisense strand of the sample alleles is labeled with a 5′-phosphorylated group and the opposing strand of the double stranded reference allele is labeled with a 5′-phosphorylated group.

42. The method of claim 5, wherein only one of the sample and reference alleles is supplied in a double stranded form and one of the strands is phosphorylated at the 5′ end.

43. The method of claim 5, wherein said reference allele is single-stranded and said sample allele is double-stranded.

44. The method of claim 5, wherein said reference allele comprises a modification which induces a mismatch between a sample and reference allele.

45. The method of claim 44, wherein at least 1% of the total number of bases in the reference allele are modified to form the modified reference allele.

46. The method of claim 6, wherein in said modified reference allele, a restriction site for at least one restriction enzyme has been removed or inactivated, and introduced at a different location.

47. The method of claim 5, wherein said duplex molecules and digested molecules are separated using separation means which physically separate molecules on the basis of one or more of size, conformation, hydrophobicity and charge.

48. The method of claim 47, wherein said separation means is selected from the group consisting of polyacrylamide gel electrophoresis (PAGE), denaturing high performance liquid chromatography (DHPLC), capillary electrophoresis and mass spectrometry.

49. The method of claim 5, wherein the sample strand of a heteroduplex formed between said sample and reference alleles is amplified following separation of said heteroduplex.

50. The method of claim 49, wherein said reference allele is further modified at a site corresponding to a primer binding site in a sample allele so as to prevent or disrupt binding of a sample amplification primer for said sample allele to said reference allele.

51. The method of claim 49, wherein a competitor primer is used to suppress amplification of the reference strand.

52. A method of genotyping the alleles present in a biological sample, comprising subjecting said sample to the method of allele separation of claim 5, separating the heteroduplexes formed between sample and reference alleles in a separation medium and identifying the alleles present, wherein the alleles present are identified by the migration pattern of the separated heteroduplexes in or on the separation medium, by direct sequencing of the sample alleles in the separated heteroduplexes, or by both the migration pattern of the separated heteroduplexes and direct sequencing of the sample alleles in the separated heteroduplexes.

53. The method of claim 52, wherein the method of genotyping the alleles present in a biological sample is used in HLA typing, determination of polymorphisms involved in metabolism of pharmaceuticals, determination of mutations in disease loci, determination of mutations in cancers, or determination of viral variants in chronic viral diseases.

54. A method of diagnosis of disease in a subject, or the susceptibility of a subject to a disease, comprising subjecting a nucleic acid sample of said subject to the method of allele separation of claim 5, and carrying out genomic typing to determine whether at least one particular mutation is present.

55. The method of claim 52, wherein the migration of the heteroduplexes in the separation medium is monitored or detected by ethidium bromide staining, detection of fluorescently-labeled alleles or by detecting elution position of column peaks.


SEQUENCE LISTING (18 sequences)

SEQ ID NO: 1 - DNA, 985 bp, Artificial
  HLA-A*0101 modified reference allele
gaaaccgcctctgtggggagaagcagggggccctcctggcgggggcgcaggaccgggggagccgcgccgggaggagggtcgggcaggtctcagccactgctcgcccccaggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgggcgccccgtggatagagcaggaggggccggagtattgggaccaggagacacggaatatgaaggcccactcacagactgaccgagcgaacctggggaccctgcgcggctactacaaccagagcgaggacggtgagtgaccccggcccggggcgcaggtcacgacccctcatcccccacggacgggccaggtcgcccacagtctccgggtccgagatccaccccgaagccgcgggactccgagacccttgtcccgggagaggcccagggcgctttacccggtttcattttcagtttaggccaaaaatccccccgggttggtcggggcggggcggggctcgggggactgggctgaccgcggggtcggggccaggttctcacaccatccagataatgtatggctgcgacgtggggccggacgggagcgccctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacgggtaccaggggccacggcgctcctccctgatcgcctatagatctcccgggctggcctcccacaagcaggggagacaattgggaccaaca

SEQ ID NO: 2 - DNA, 985 bp, Homo sapiens
  HLA-A*0101allele
gaaaccgcctctgcggggagaagcaaggggccctcctggcgggggcgcaggaccgggggagccgcgccgggaggagggtcgggcaggtctcagccactgctcgcccccaggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggtgagtgaccccggcccggggcgcaggtcacgacccctcatcccccacggacgggccaggtcgcccacagtctccgggtccgagatccaccccgaagccgcgggactccgagacccttgtcccgggagaggcccaggcgcctttacccggtttcattttcagtttaggccaaaaatccccccgggttggtcggggcggggcggggctcgggggactgggctgaccgcggggtcggggccaggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacgggtaccaggggccacggggcgcctccctgatcgcctatagatctcccgggctggcctcccacaaggaggggagacaattgggaccaaca

SEQ ID NO: 3 - DNA, 26 bp, Artificial
  primer HLA-AF
gaaacsgcctctgyggggagaagcaa

SEQ ID NO: 4 - DNA, 24 bp, Artificial
  primer HLA- AR
tgttggtcccaattgtctcccctc

SEQ ID NO: 5 - DNA, 20 bp, Artificial
  primer HLA-ClI-ex3
acccggtttcattttcrgtt

SEQ ID NO: 6 - DNA, 20 bp, Artificial
  HLA-clI-ex2 primer
aacygaaaatgaaaccgggt

SEQ ID NO: 7 - DNA, 32 bp, Artificial
  MISC_FEATURE (6)..(6): n is a, c, t or g
gaaacngcctctgyggggagaagcagcgggcc

SEQ ID NO: 8 - DNA, 30 bp, Artificial
  HLA-AR ref primer
tgttggtcccaattgtctcccctgcttgtg

SEQ ID NO: 9 - DNA, 26 bp, Artificial
  MISC_FEATURE (1)..(12): n is a, c, t or g
nnnnnnnnnnnnggggagaagcadda

SEQ ID NO: 10 - DNA, 26 bp, Artificial
  MISC_FEATURE (1)..(12): n is a, c, t or g
nnnnnnnnnnnnttgtctcccctddc

SEQ ID NO: 11 - DNA, 32 bp, Artificial
  hlaa-p250F primer
gaagatggagccgggcgccccgtggatagagc

SEQ ID NO: 12 - DNA, 32 bp, Artificial
  hlaa-p250R primer
gctctatccacggggcgcccggctccatcttc

SEQ ID NO: 13 - DNA, 37 bp, Artificial
  hlaa-p500F primer
aagatggagccgggcgccccgtggatagagcattttc

SEQ ID NO: 14 - DNA, 37 bp, Artificial
  primer hlaa-p500R
gaaaatgaaaccgggtaaagcgccctgggcctctccc

SEQ ID NO: 15 - DNA, 26 bp, Artificial
  hlaa-p650F primer
gggccggaagcgctcttcctccgcgg

SEQ ID NO: 16 - DNA, 26 bp, Artificial
  hlaa-p650R primer
ccgcggaggaagagcgcttccggccc

SEQ ID NO: 17 - DNA, 29 bp, Artificial
  hlaa-p910F primer
caggggccacggcgctcctccctgatcgc

SEQ ID NO: 18 - DNA, 29 bp, Artificial
  hlaa-p910R primer
gcgatcagggaggagcgccgtggcccctg